SEQUENCE LISTING (23 sequences)

SEQ ID NO: 1 - protein, 246 aa, Staphylococcus simulans
  source 1..246: note = Lysostaphin
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 2 - DNA, 22 bp, synthetic construct
  source 1..22: mol_type = other DNA; note = P438 promoter
tagtatttagaattaataaagt

SEQ ID NO: 3 - DNA, 150 bp, synthetic construct
  source 1..150: mol_type = other DNA; note = EfTu promoter
gaagaccttttgtgctaacgccagtttggcaaatcaagttctgattttgcaattattttgctccatatgaattacactactccaagaattataagcctctctacagctttatctcaaacttatgtaaaattagagacgtaattcaaacac

SEQ ID NO: 4 - DNA, 39 bp, synthetic construct
  misc_feature 1..39: note = P1 promoter
  source 1..39: mol_type = other DNA
ttcccaaattttcgaaataatattgtataattaaaaata

SEQ ID NO: 5 - DNA, 39 bp, synthetic construct
  misc_feature 1..39: note = P2 promoter
  source 1..39: mol_type = other DNA
tattgcataatattgcataatattgtataattaaaaata

SEQ ID NO: 6 - DNA, 39 bp, synthetic construct
  misc_feature 1..39: note = P3 promoter
  source 1..39: mol_type = other DNA
tattgcataatattgtataatattgtataattaaaaata

SEQ ID NO: 7 - DNA, 39 bp, synthetic construct
  misc_feature 1..39: note = P4 promoter
  source 1..39: mol_type = other DNA
tattgtataatattgtataatattgtataattaaaaata

SEQ ID NO: 8 - DNA, 75 bp, synthetic construct
  misc_feature 1..75: note = P5 promoter
  source 1..75: mol_type = other DNA
ttcccaaattttcgaaataatattgtataattaaaaatatattgtataattaaaaatattgtataattaaaaata

SEQ ID NO: 9 - DNA, 63 bp, synthetic construct
  misc_feature 1..63: note = Psyn promoter
  source 1..63: mol_type = other DNA
gttaacgattaagatcaaaaagtgcctggtatcgtaaataatattgtataattaaaaaagaat

SEQ ID NO: 10 - protein, 31 aa, synthetic construct
  REGION 1..31: note = Optimised MPN142 secretion signal
MKSKLKLKRYLLFLPLLPLGTLSLANTYLLQ

SEQ ID NO: 11 - protein, 247 aa, synthetic construct
  REGION 1..247: note = Truncated lysostaphin
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 12 - protein, 33 aa, synthetic construct
  REGION 1..33: note = MPN036 secretion signal
MKKMTLHKLLLGLFLPAILGPLFGLLVTKTDAE

SEQ ID NO: 13 - protein, 28 aa, synthetic construct
  REGION 1..28: note = MPN142 secretion signal
MKSKLKLKRYLLFLPLLPLGTLSLANTY

SEQ ID NO: 14 - protein, 28 aa, synthetic construct
  REGION 1..28: note = MPN645 secretion signal
MKLKLKFLLISLLGSSLLLSACSSAATQ

SEQ ID NO: 15 - protein, 38 aa, synthetic construct
  REGION 1..38: note = MPN400 secretion signal
MKLNFKIKDKKTLKRLKKGGFWALGLFGAAINAFSAVL

SEQ ID NO: 16 - protein, 23 aa, synthetic construct
  REGION 1..23: note = MPN200 secretion signal
MKFKYGAIVFSGLLGVSAILAAC

SEQ ID NO: 17 - protein, 30 aa, synthetic construct
  REGION 1..30: note = MPN213 secretion signal
MKLSAIISLSVAGTVGTTAVWPTTITLVNK

SEQ ID NO: 18 - protein, 31 aa, synthetic construct
  REGION 1..31: note = MPN489 secretion signal
MGYKLKRWPLVAFTFTGIGLGVVLAACSALN

SEQ ID NO: 19 - protein, 381 aa, synthetic construct
  source 1..381: note = Dispersin B
MNYIKKIILSLFLLGLFSVLNCCVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 20 - protein, 229 aa, synthetic construct
  source 1..229: note = Alginate lyase AI-II'
MPAAAPGKNFDLSHWKLQLPDANTTEISSANLGLGYTSQYFYTDTDGAMTFWAPTTGGTTANSSYPRSELREMLDPSNSKVNWGWQGTHTMKLSGKTVQLPSSGKIIVAQIHGIMDDGTNAPPLVKAVFQDGQLDMQVKQNSDGTGSDVHNYFTGIKLGDLYNMEIRVTDGVAYVTMNGDTRSVDFVGKDAGWKNLKYYFKAGNYVQDNTSTGGSAIAKLYSLSVSHSN

SEQ ID NO: 21 - protein, 258 aa, synthetic construct
  source 1..258: note = PelAh
MGGPSSVAFWYAERPPLAELSQFDWVVLEAAHLKPADVGYLKEQGSTPFAYLSVGEFDGDAAAIADSGLARGKSAVRNQAWNSQVMDLAAPSWRAHLLKRAAELRKQGYAGLFLDTLDSFQLQAEERREGQRRALASFLAQLHRQEPGLKLFFNRGFEVLPELPGVASAVAVESIHAGWDAAAGQYREVPQDDRDWLKGHLDALRAQGMPIVAIDYLPPERRDEARALAARLRSEGYVPFVSTPALDYLGVSDVEVQP

SEQ ID NO: 22 - protein, 413 aa, synthetic construct
  REGION 1..413: note = PslGh
MEIQVLKAPRAVVWKDFLGVNAQFLWFSPERYNKQIDRLQDLGLEWVRLDLHWDRLETAEDQYQLASLDQLVKDLEARQLKSVFYLVGSARFITTAPFYSPFQDQYPPRDPEVFARRMAMLSQRYPSVAAWQVWNEPNLIGFWRPKADPEGYAKLLQASTIALRMVDPEKPVVSAGMAFFSEMPDGRTMFDALGHLGVESLGTIATYHPYTQLPEGNYPWNLDFVSHANQINRALRNAGVPAIWSTEWGWSAYKGPKELQDIIGVEGQADYVLRRLALMSALDYDRIFLFTLSDLDQRASVRDRDYGLLDLDANPKPVYLALQRFLKVTGPKLRPADPPVTEDLPDGSFSIGWTREDGRNVWLFWSARGGNVRLPKLKEATLHDPLSGKVTPLSGSDGLEVPVKSSLQMLVWE

SEQ ID NO: 23 - DNA, 93 bp, synthetic construct
  misc_feature 1..93: note = Optimised MPN142 secretion signal
  source 1..93: mol_type = other DNA
atgaagtccaagttgaaactcaaacgctatttactctttctccccttgttaccactcggtaccttgagtttagctaacacttacttgttacaa

(19) United States
(12) Patent Application Publication
(10) Pub. No.: US 2024/0382537 A1
(43) Pub. Date: Nov. 21, 2024

(54) Genetically Modified Mycoplasma Bacteria Active Against Heterogenous Bacterial Biofilms

(71) Applicants: Pulmobiotics, S.L., Barcelona (ES); Fundació Centre De Regulació Genòmica, Barcelona (ES); Institució Catalana De Recerca I Estudis Avançats, Barcelona (ES)

(72) Inventors: Rocco Mazzolini, Barcelona (ES); Maria Lluch Senar, Sant Cugat del Vallés, Barcelona (ES); Claudio Joao Ribeiro Dos Santos, Salamanca (ES); Luis Serrano Pubul, Barcelona (ES)

(21) Appl. No.: 18/693,284

(22) PCT Filed: Sep. 20, 2022
(86) PCT No.: PCT/EP2022/076135

§ 371 Date: Mar. 19, 2024

(30) Foreign Application Priority Data
Sep. 20, 2021 (EP) 21382847.8

(51) Int. Cl. A61K 35/74; A61P 31/04; C12N 1/36; C12N 9/52; C12N 15/90

(52) U.S. Cl. CPC A61K 35/74; A61P 31/04; C12N 1/36; C12N 9/52; C12N 15/902; C12Y 304/24075

(57) ABSTRACT

The present invention concerns genetically modified Mycoplasma bacteria that express gene products having an anti-biofilm activity against a biofilm formed by multiple bacteria such as Pseudomonas aeruginosa and Staphylococcus aureus. Further intended are pharmaceutical compositions comprising genetically modified Mycoplasma bacteria and their use as a medicament.

### FIELD OF THE INVENTION

This application is a national phase entry under 35 U.S.C. § 371 of International Patent Application PCT/EP2022/076135, filed Sep. 20, 2022, designating the United States of America and published in English as International Patent Publication WO 2023/041809 on Mar. 23, 2023, which claims the benefit under Article 8 of the Patent Cooperation Treaty to European Patent Application Serial No. 21382847.8, filed Sep. 20, 2021, the entireties of which are hereby incorporated by reference.

### INCORPORATION BY REFERENCE

The ST.26 XML Sequence listing named “10379 US Sequence listing ST26”, created on Sep. 13, 2022, and having a size of 32.768 bytes, is hereby incorporated herein by this reference in its entirety.

### FIELD OF THE INVENTION

The invention relates to the field of molecular biology, more specifically the field of genome engineering and synthetic biology. Aspects of the invention relate to a genetically reprogrammed Mycoplasma bacterium for use in dissolving biofilms caused by bacteria in the respiratory tract, particularly biofilms generated by a group of bacteria comprising or consisting essentially of Pseudomonas aeruginosa and/or Staphylococcus aureus.

### BACKGROUND OF THE INVENTION

Biofilms are complex and dynamic structures formed by different pathogens that cause chronic, persistent and recurrent infections. In biofilms, the adherent cells become embedded within a slimy extracellular matrix that is composed of extracellular polymer substances comprising amongst other components exopolysaccharides. It is estimated that approximately 65-80% of human infections are associated with biofilm formation (Jamal et al., Bacterial biofilm and associated infections, Journal of the Chinese Medical Association, 2018). Pathogenic biofilms are especially frequent in pulmonary infectious diseases, like cystic fibrosis (CF), chronic obstructive pulmonary disease (COPD), bronchiectasis and ventilator-associated pneumonia (VAP) (Boisvert et al., Microbial biofilms in pulmonary and critical rare diseases, Annals of the American Thoracic Society, 2016).

One of the major challenges in treating biofilm infections is the increased resistance of the bacteria within the biofilm to antimicrobial agents and host defense mechanisms (Høiby et al., Antibiotic resistance of bacterial biofilms, International Journal of Antimicrobial Agents, 2010). Metabolic activity of bacterial cells is low, resulting in many slow-growing cells. Thus, cell division occurs at radically down-regulated rates. Therefore, antibiotics such as β-lactams which are only active against dividing cells are not efficient at eradicating biofilm infections (Amanatidou et al., Biofilms facilitate cheating and social exploitation of β-lactam resistance in Escherichia coli, Biofilms and Microbiomes, 2019). These diseases are very difficult to manage therapeutically, as the effective antibiotic minimum bactericidal concentration for biofilm eradication in vivo are impossible to reach without causing adverse effects and renal and/or hepatic injury with numerous pathogenic bacterial strains being even antibiotic resistant. Furthermore, the development of efficient anti-biofilm treatment strategies is hampered by numerous polymicrobial interactions that affect the taxonomic and structural composition of the resulting biofilm, which lead to biofilm behavior and characteristics that differ significantly from those of the individual constituent species and hence in clinical importance (Limoli and Hoffman, Help, hinder, hide and harm: what can we learn from the interactions between Pseudomonas aeruginosa and Staphylococcus aureus during respiratory infections?, Thorax, 2019). Thus, in vitro interactions do not always reflect in vivo behaviors of biofilms. Finally, given the heterogeneity in bacterial composition of these biofilms, there is a general paucity of traditional antibiotics that are able to effectively counter different spatial areas of the biofilm, which, as mentioned above, may comprise markedly different bacteria and may thus also act as a seed area for reconstitution of the biofilm.

Engineering bacteria provides several advantages as a therapy delivery vehicle compared with simple drugs, nanoparticles or phages: i) they contain all biological machinery needed to synthesize complex therapeutics; ii) complex regulatory circuits can be integrated into bacteria to sense and to respond specifically to diseased tissue; iii) there is a low risk of bacterial DNA integration into the host genome; iv) in most cases, bacteria proliferation can be effectively controlled by using antibiotics as contingency strategy; and v) killing circuits or auxotrophic dependence modules can be engineered to control their growth for biocontainment and biosafety.

Nevertheless, a major bottleneck towards utilizing bacteria for (human) therapy is the difficulty to predict the behavior of engineered bacteria in host organisms to which said bacteria are introduced which may differ considerably from their behavior in well-controlled in vitro and/or ex vivo conditions. In addition, several bacterial strains that prima facie appear interesting have been historically difficult to engineer due to a lack of genetic tools. Furthermore, the niche or site of action is one of the multiple factors to consider for bacterial therapeutics. Ideally, a bacterium should be used that is naturally present in the organ to be treated, to ensure the survival of the bacterium and to limit its spreading to other organs. For example, although a previous study reported on the engineering of a E. coli Nissle 1917 strain to treat P. aeruginosa infections in the gut, it cannot be used to treat respiratory infections as the respiratory tract is not its natural niche (De Smet et al., Pseudomonas predators: understanding and exploiting phage-host interactions, Nat Rev Microbiol, 2017). Finally, numerous research groups have reported on perturbations in various aspects of the metabolism of bacteria that are altered with the aim of expressing recombinant proteins. These perturbations are typically caused by e.g., drainage of the metabolic resources of the bacterium (e.g., increased metabolic burden), formation of inclusion bodies due to unfolded or partially folded protein aggregates of the recombinant proteins, blockage of one or more translocation pathways, and combinations thereof.

Thus, despite earlier progress in the technical field, there remains an unmet need for (improved) engineered bacteria that are able to efficiently combat (i.e., counteract, disperse) a clinically relevant biofilm characterized by a heterogenous bacterial composition. Furthermore, these engineered bacteria should not be afflicted by an increased metabolic load and should be able to maintain their ability to propagate and survive in proximity of the biofilm, enabling a prolonged and/or sustained anti-biofilm activity.

### SUMMARY OF THE INVENTION

The inventors provide a new and innovative approach to treat biofilms formed by bacterial pathogens. In particular, the inventors have discovered that genetically reprogrammed bacteria such as but not limited to Mycoplasma bacteria are suited for avoiding biofilm formation and/or dissolving biofilms formed by multiple bacteria, in a host organism. Hence, the genetically modified bacteria or pharmaceutical dosage units comprising said bacteria are particularly effective against bacterial biofilms characterized by a considerable degree of heterogeneity. Generation of such bacteria have now become feasible due to advances in the field of bacterial genome engineering, to which the inventors made considerable contributions (e.g., as described in PCT/EP2021/052110). In particular, M. pneumoniae strains have been developed that are effective against Pseudomonas aeruginosa and Staphylococcus aureus biofilms and which are at the same time able to replicate in the respiratory tract without any impediments that may be caused by increased metabolic load and most importantly without causing unwanted side-effects (such as but not limited to disease images) in the subject (i.e. patient). The invention further concerns pharmaceutical dosage units and compositions comprising a reprogrammed (i.e. genetically modified) Mycoplasma bacterium as described herein. Hence, in response to the clear need that is formulated in the state of the art, the invention provides a new strategy to treat pulmonary infections associated with biofilm formation caused by multiple bacteria.

The invention therefore provides the following numbered aspects:

Aspect 1. A genetically modified Mycoplasma bacterium comprising in its genome a deletion, substitution, and/or insertion of one or more nucleotides in the operons of the Ca2+ dependent cytotoxic nuclease gene (MPN133) or orthologues thereof and ADP-ribosyltransferase CARDS gene (MPN372) or orthologues thereof, that reduce the pathogenicity and/or immunogenicity of said Mycoplasma bacterium compared to a reference M129-B7 Mycoplasma pneumoniae bacterium, said reduction in pathogenicity and/or immunogenicity being characterized by a reduction of toxicity by at least 30% upon introduction into a host organism when compared to reference M129-B7 Mycoplasma pneumoniae bacterium,
wherein said Mycoplasma bacterium further comprises in its genome an oligonucleotide arrangement, said oligonucleotide arrangement comprising:
   i) a first nucleotide sequence encoding a first heterologous exopolysaccharide hydrolyzing enzyme under the control of a promoter or a functional variant of said promoter or fragment thereof which is active in said Mycoplasma bacterium, and wherein said exopolysaccharide hydrolyzing enzyme is Dispersin B, and
   ii) at least one further nucleotide sequence encoding further heterologous exopolysaccharide hydrolyzing enzymes under the control of a promoter or a functional variant of said promoter or fragment thereof which is active in said Mycoplasma bacterium, and wherein said further exopolysaccharide hydrolyzing enzymes are selected from the group consisting of: Alginate lyase AI-II′, PelAh, PslGh, or any combination thereof.

Aspect 2. The genetically modified Mycoplasma bacterium according to aspect 1, wherein said bacterium further comprises one or more antimicrobial agents not encoded by or expressed in a Mycoplasma pneumoniae bacterium, preferably wherein the one or more antimicrobial agents are encoded by the genome of said genetically modified Mycoplasma bacterium, more preferably encoded in a further nucleotide sequence under the control of a promoter or a functional variant of said promoter or fragment thereof which is active in said Mycoplasma bacterium.

Aspect 3. A pharmaceutical dosage unit comprising:

1) a genetically modified Mycoplasma bacterium comprising in its genome a deletion, substitution, and/or insertion of one or more nucleotides in the operons of the Ca2+dependent cytotoxic nuclease gene (MPN133) or orthologues thereof and ADP-ribosyltransferase CARDS gene (MPN372) or orthologues thereof, that reduce the pathogenicity and/or immunogenicity of said Mycoplasma bacterium compared to a reference M129-B7 Mycoplasma pneumoniae bacterium, said reduction in pathogenicity and/or immunogenicity being characterized by a reduction of toxicity by at least 30% upon introduction into a host organism when compared to a reference M129-B7 Mycoplasma pneumoniae bacterium,
wherein said Mycoplasma bacterium further comprises in its genome an oligonucleotide arrangement, said oligonucleotide arrangement comprising:
   i) a first nucleotide sequence encoding a first heterologous exopolysaccharide hydrolyzing enzyme under the control of a promoter or a functional variant of said promoter or fragment thereof which is active in said Mycoplasma bacterium, and wherein said exopolysaccharide hydrolyzing enzyme is Dispersin B, and
   ii) at least one further nucleotide sequence encoding further heterologous exopolysaccharide hydrolyzing enzymes under the control of a promoter or a functional variant of said promoter or fragment thereof which is active in said Mycoplasma bacterium, and wherein said further exopolysaccharide hydrolyzing enzymes are selected from the group consisting of: Alginate lyase AI-II′, PelAh, PslGh, or any combination thereof; and

2) One or more antimicrobial agents not encoded by or expressed in a reference Mycoplasma pneumoniae bacterium.

A skilled person appreciates that Aspect 3 may be alternatively worded as a pharmaceutical dosage unit comprising
1) the genetically modified bacterium according to aspect 1, and
2) one or more antimicrobial agents not encoded by or expressed in a reference Mycoplasma pneumoniae bacterium.

Aspect 4. The genetically modified Mycoplasma bacterium according to aspect 2 or the pharmaceutical dosage unit according to aspect 3, wherein the antimicrobial agent is a bacteriocin, an antimicrobial peptide, an antibiotic, a defensin, or any combination thereof.

Aspect 5. The genetically modified Mycoplasma bacterium or the pharmaceutical dosage unit according to aspect 4, wherein the bacteriocin is a gram negative bacteriocin, a gram positive bacteriocin, or any combination thereof.

Aspect 6. The genetically modified Mycoplasma bacterium or the pharmaceutical dosage unit according to aspect 5, wherein the gram negative bacteriocin is selected from the group consisting of: microcins, colicin-like bacteriocins, and tailocins.

Aspect 7. The genetically modified Mycoplasma bacterium or the pharmaceutical dosage unit according to aspect 5, wherein the gram positive bacteriocin is selected from the group consisting of: class I bacteriocins, class II bacteriocins, class III bacteriocins, and class IV bacteriocins.

Aspect 8. The genetically modified Mycoplasma bacterium or the pharmaceutical dosage unit according to aspect 4, wherein the bacteriocin is selected from the group consisting of: acidocin, actagardine, agrocin, alveicin, aureocin, aureocin A53, aureocin A70, bisin, carnocin, carnocyclin, caseicin, cerein, circularin A, colicin, curvaticin, divercin, duramycin, enterocin, enterolysin, epidermin, gallidermin, erwiniocin, gardimycin, gassericin A, glycinecin, halocin, haloduracin, klebicin, lactocin S, lactococcin, lacticin, leucoccin, lysostaphin, macedocin, mersacidin, mesentericin, microbisporicin, microcin, microcin S, mutacin, nisin, paenibacillin, planosporicin, pediocin, pentocin, plantaricin, pneumocyclicin, pyocin, reutericin 6, reutericyclin, reuterin, sakacin, salivaricin, sublancin, subtilin, sulfolobicin, tasmancin, thuricin 17, trifolitoxin, variacin, vibriocin, warnericin, warnerin, or any combination thereof, preferably wherein the bacteriocin is lysostaphin.

Aspect 9. The genetically modified Mycoplasma bacterium or the pharmaceutical dosage unit according to aspect 4, wherein the antimicrobial agent is a defensin, preferably a defensin selected from the group consisting of alpha (α)-defensins, beta (β)-defensins, theta(θ)-defensins, defensin-like peptides, or any combination thereof, more preferably wherein the defensin is a human a-defensin or human β-defensin.

Aspect 10. The genetically modified Mycoplasma bacterium or the pharmaceutical dosage unit according to aspect 9, wherein the α-defensin is a human α-defensin, preferably a human α-defensin selected from the group of consisting of: neutrophil defensin 1, defensin alpha 1, neutrophil defensin 3,neutrophil defensin 4, defensin-5, defensin-6, or any combination thereof.

Aspect 11. The genetically modified Mycoplasma bacterium or the pharmaceutical dosage unit according to aspect 9, wherein the defensin is a trans-defensin or cis-defensin.

Aspect 12. The genetically modified Mycoplasma bacterium or the pharmaceutical dosage unit according to aspect 4, wherein the antimicrobial agent is an antimicrobial peptide selected from the group consisting of membranolytic antimicrobial peptides, non-membranolytic antimicrobial peptides, or any combination thereof.

Aspect 13. The genetically modified Mycoplasma bacterium or the pharmaceutical dosage unit according to aspect 4, wherein the antimicrobial agent is an antimicrobial peptide selected from the group consisting of: Bacitracin, Dalbavancin, Daptomycin, Oritavancin, Teicoplanin, Telavancin, Vancomycin, Guavanin 2, Cecropin A, or any combination thereof.

Aspect 14. The genetically modified Mycoplasma bacterium or the pharmaceutical dosage unit according to aspect 4, wherein the antimicrobial agent is an antibiotic, preferably wherein the antibiotic is a bacterial membrane targeting antibiotic, more preferably wherein the antibiotic is selected from the group consisting of: Piperacillin, Tazobactam, Ciprofloxacin, Levofloxacin, Meropenem, Imipenem, Cilastatin, Amikacin, Ceftazidime, Avibactam, Ceftolozane, Ceftriaxone, Vancomycin, Linezolid, or any combination thereof.

Aspect 15. The genetically modified Mycoplasma bacterium or the pharmaceutical dosage unit according to any one or aspects 1 to 14 wherein said further heterologous exopolysaccharide hydrolyzing enzymes are Alginate lyase AI-II', PelAh and PslGh, under the control of at least one promoter or a functional variant of said promoter or fragment thereof which is active in said Mycoplasma bacterium.

Aspect 16. The genetically modified Mycoplasma bacterium or the pharmaceutical dosage unit according to any one of aspects 1 to 15 wherein each of said further heterologous exopolysaccharide hydrolyzing enzymes is under the control of a promoter or a functional variant of said promoter or fragment thereof which is active in said Mycoplasma bacterium.

Aspect 17. The genetically modified Mycoplasma bacterium or the pharmaceutical dosage unit according to any one of aspects 1 to 16, wherein at least one nucleotide sequence comprises a synthetic promoter with a nucleotide sequence of at least 65% identity, preferably at least 75% identity, more preferably at least 85% identity, most preferably at least 95% identity to the nucleotide sequence of SEQ ID NO: 6.

Aspect 18: The genetically modified Mycoplasma bacterium or the pharmaceutical dosage unit according to aspect 8, wherein expression of lysostaphin is controlled by a promoter sequence comprising a nucleotide sequence of at least 65% identity, preferably at least 75% identity, more preferably at least 85% identity, even more preferably at least 95% identity to the nucleotide sequence of SEQ ID NO: 6, most preferably wherein expression of lysostaphin is controlled by a promoter sequence having 100% sequence identity to SEQ ID NO: 6.

Aspect 19. The genetically modified Mycoplasma bacterium or the pharmaceutical dosage unit according to any one of aspects 1 to 18, wherein the first heterologous exopolysaccharide hydrolyzing enzymes is under the control of the EfTu promoter defined by SEQ ID NO: 3, and the further heterologous exopolysaccharide hydrolyzing enzymes are under the control of the EfTu promoter defined by SEQ ID NO: 3.

Aspect 20. The genetically modified Mycoplasma bacterium or the pharmaceutical dosage unit according to any one of aspects 1 to 19, wherein the Mycoplasma bacterium has a genomic sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, at least 97% global sequence identity to a naturally occurring Mycoplasma bacterium, preferably at least 30%, at least 35%, at least 40%, at least 45%, at least 50%, at least 55%, at least 60%, at least 65%, at least 70%, at least 75%, at least 80%, at least 85%, at least 90%, at least 95%, at least 97% global sequence identity to reference M129-B7 Mycoplasma pneumoniae bacterium.

Aspect 21. The genetically modified Mycoplasma bacterium or the pharmaceutical dosage unit according to any one of aspects 1 to 20, wherein the Mycoplasma bacterium is Mycoplasma pneumoniae.

Aspect 22. The genetically modified Mycoplasma bacterium or the pharmaceutical dosage unit according to any one of aspects 1 to 21, wherein the first exopolysaccharide hydrolyzing enzyme is active against Pseudomonas aeruginosa and the at least one further exopolysaccharide hydrolyzing enzyme is active against Staphylococcus aureus.

Aspect 23. The genetically modified Mycoplasma bacterium or the pharmaceutical dosage unit according to any one of aspects 1 to 22, which is active against biofilms that are heterogenous in bacterial composition.

Aspect 24. The genetically modified Mycoplasma bacterium or the pharmaceutical dosage unit according to aspect 23, wherein the biofilm is characterised by the presence of Pseudomonas aeruginosa and Staphylococcus aureus.

Aspect 25. The genetically modified Mycoplasma bacterium or the pharmaceutical dosage unit according to aspects 23 or 24, wherein the biofilm is an endotracheal tube biofilm, preferably an in vivo endotracheal tube biofilm.

Aspect 26. The genetically modified Mycoplasma bacterium or the pharmaceutical dosage unit according to any one of aspects 1 to 25, wherein the genetically modified Mycoplasma bacterium is a live genetically modified Mycoplasma bacterium, more preferably a live genetically modified Mycoplasma pneumoniae bacterium.

Aspect 27. The pharmaceutical dosage unit according to any one of aspects 3 to 26, wherein the genetically modified Mycoplasma bacterium is comprised in a first composition and the one or more antimicrobial agents are comprised in at least a further composition.

Aspect 28. The pharmaceutical dosage unit according to any one of aspects 3 to 27, wherein the genetically modified Mycoplasma bacterium and the one or more antimicrobial agents are comprised in a single composition.

Aspect 29. A genetically modified Mycoplasma bacterium or a pharmaceutical dosage unit according to any one of aspects 1 to 28 for use as a medicament.

Aspect 30. A genetically modified Mycoplasma bacterium or a pharmaceutical dosage unit according to any one of aspects 1 to 29 for use in treating pneumonia.

Aspect 31. A genetically modified Mycoplasma bacterium or a pharmaceutical dosage unit according to any one of aspects 1 to 30 for use in treating ventilator-associated pneumonia (VAP).

Aspect 32. A genetically modified Mycoplasma bacterium or a pharmaceutical dosage unit according to any one of aspects 1 to 31 for use in dissolving microbial biofilms produced by and/or comprising Pseudomonas aeruginosa and Staphylococcus aureus.

Aspect 33. Use of a genetically modified Mycoplasma bacterium or a pharmaceutical dosage unit according to any one of aspects 1 to 32 for avoiding biofilm formation and/or for dissolving biofilms.

Aspect 34. Use of a genetically modified Mycoplasma bacterium or a pharmaceutical dosage unit according to any one of aspects 1 to 33 for the manufacture of a medicament for the prevention or treatment of a pathogenic biofilm, preferably for the prevention or treatment of a respiratory tract biofilm.

Aspect 35. A method of treating a respiratory infection in a subject in need thereof, comprising administering the genetically modified Mycoplasma bacterium or the pharmaceutical dosage unit according to any one of aspects 1 to 34 to said subject.

Aspect 36. The method according to aspect 35, wherein the genetically modified Mycoplasma bacterium and the one or more antimicrobial agents are administered simultaneously.

Aspect 37. The method according to aspect 36, wherein the genetically modified Mycoplasma bacterium of the pharmaceutical dosage unit and the one or more antimicrobial agents of the pharmaceutical dosage unit are administered at distinct time points.

Aspect 38. The method according to aspect 37, wherein the one or more antimicrobial agents of the pharmaceutical dosage unit are administered at least at two distinct time points.

Aspect 39. Use of an antimicrobial agent for increasing the in vivo potency of an attenuated bacterium as a therapeutic agent.

Aspect 40. The use according to aspect 39, wherein the antimicrobial agent is a bacteriocin, an antimicrobial peptide, an antibiotic, or any combination thereof.

Aspect 41. The use according to aspect 39 or 40, wherein the attenuated bacterium is a bacterium wherein one or more virulence factors are no longer functionally expressed.

Aspect 42. The use according to any one of aspects 39 to 41, wherein the attenuated bacterium is a Mycoplasma bacterium, preferably a Mycoplasma pneumoniae bacterium.

Aspect 43. The use according to any one of aspects 39 to 42, wherein the in vivo potency of the attenuated bacterium is increased by at least about 30%, preferably by at least about 40%, preferably by at least about 50%, preferably by at least about 60%, preferably by at least about 70%, more preferably by at least about 80%, most preferably by at least about 90% when compared to the corresponding genetically modified bacterium not containing, not in presence of, or not supplemented with the heterologous antimicrobial agent.

Aspect 44. The use according to any one of aspects 39 to 43, wherein the in vivo potency is restored to about the potency of the corresponding non-attenuated reference bacterium.

Aspect 45. The use according to any one of aspects 39 to 44, wherein the one or more virulence factors are Ca2+ dependent cytotoxic nuclease gene (MPN133) or orthologues thereof, and/or ADP-ribosyltransferase CARDS gene (MPN372) or orthologues thereof.

Aspect 46. The use according to any one of aspects 39 to 45, wherein the in vivo potency of the attenuated bacterium as a therapeutic agent is the in vivo potency of the attenuated bacterium to act as a therapeutic agent against bacterial infections, preferably bacterial infections of the respiratory tract.

Aspect 47. The use according to aspect 46, wherein the bacterial infection is characterised by biofilm formation, preferably characterised by biofilm formation due to the presence of Pseudomonas aeruginosa and/or Staphylococcus aureus, preferably Staphylococcus aureus.

Aspect 48. The use according to any one of aspects 39 to 47, wherein the antimicrobial agent is lysostaphin.

Aspect 49. The use according to aspect 48, wherein the lysostaphin has a sequence identity of at least 65%, preferably at least 75%, more preferably at least 85%, most preferably at least 95% to SEQ ID NO: 11.

The above and further aspects and preferred embodiments of the invention are described in the following sections and in the appended claims. The subject matter of the appended claims is hereby specifically incorporated in this specification.

### BRIEF DESCRIPTION OF THE FIGURES

FIGS. 1A and 1B. In vitro dispersion assay of S. aureus mature biofilms formed in microplates. (FIGS. 1A,1B) Mature S. aureus biofilms were allowed to develop for 24 h in polystyrene plates and then treated for the indicated time intervals with cell suspensions, or culture supernatants of the CV2 or the CV2-DispB strain (FIG. 1A) or the WT or the WT-DispB strain (FIG. 1B). Biofilm presence was assessed by crystal violet staining and included a negative staining control without biofilm. The results are expressed as mean ±s.d. of OD 595 nm absorbance values from three biological replicates (n=3). Results from Fisher's Post-hoc Least Significant Difference (PLSD) test are shown for those treatments statistically different from the control treatment (Hayflick medium). *P≤0.05; **P≤0.005; ***P≤0.0005; ****P≤0.00005.

FIGS. 2A-2D. In vitro and ex vivo dispersion assays of S. aureus mature biofilms formed on catheters. (FIG. 2A) Schematic representation of the experimental procedure. Catheters pre-colonized with S. aureus were allowed to form biofilms by incubation at 37° C. (for in vitro dispersion assay) or by subcutaneous implantation in mice (for ex vivo dispersion assay) before being treated in vitro with different Mycoplasma strains and estimate biofilm dispersion by crystal violet staining. (FIG. 2B) Representative pictures of catheters from the ex vivo dispersion assay with the indicated strains after crystal violet staining are shown. A staining control based on catheters in which no biofilm is formed was also included. (FIGS. 2C-2D) Plots showing results obtained from the in vitro or ex vivo dispersion assays with the indicated strains and from the staining control. Each circle represents the OD 595 nm values obtained for individual catheters (n≥4), whereas mean ±s.d. is represented with lines inside cach group. Results from Fisher's PLSD test are shown for treatments statistically different from those based on strains not secreting dispersin B (i.e., the WT or CV2 strains). *P≤0.05; **P≤0.005; ***P<0.0005.

FIGS. 3A-3D. In vivo dispersion assay of S. aureus mature biofilms formed on catheters. (FIG. 3A) Schematic representation of the experimental procedure. Catheters pre-colonized with S. aureus were allowed to form biofilms in an in vivo context by subcutaneous implantation in mice. 24 h post-implantation mice were treated with a single subcutaneous injection of different Mycoplasma strains and the effectiveness of each treatment was followed by positron tomography with [¹⁸F]-FDG-MicroPET. (FIG. 3B) Representative images of longitudinal slices of [¹⁸F]-FDG-MicroPET uptake in mice carrying implanted catheters (arrows) at day 1 or day 4 of the treatments. Micro-PET images have been superimposed with CT-3D images used as anatomical reference. Brain location is highlighted (b). (FIGS. 3C. 3D) Plots showing the SUV60 variation (%) between day 1 and day 4 of the different treatments. Each circle represents the SUV60 variation obtained for individual animals (n≥4), whereas mean±s.d. is represented with lines inside each group. Data below the dotted lines indicate that the SUV 60 values decreased at D4 post-treatment. Results from Fisher's PLSD test are shown. *P≤0.05;**P≤0.005; ***P≤0.0005.

FIGS. 4A-4D. Representative three-dimensional (3D) volume reconstructions of optical coherence tomography (OCT) and relative attenuation coefficient (RAC) images along the entire length of the endotracheal tube (ETT) (straightened for visualization purposes). (FIG. 4A) 3D OCT and (FIG. 4B) RAC images from an ETT that was negative for biofilm (Subject 6. Day 3). (FIG. 4C) 3-D OCT and (FIG. 4D) RAC images from an ETT positive for biofilm (Subject 2. Day 6). Increased optical attenuation from the biological structures within the ETT tube identifies the presence and 3D distribution of biofilm throughout the length of the ETT. Figure taken from Dsouza et al. . . . In vivo detection of endotracheal tube biofilms in intubated critical care patients using catheter-based optical coherence tomography. Journal of Biophotonics. 2019.

FIGS. 5A and 5B. Expression of heterologous proteins by different M. pneumoniae strains. (FIG. 5A) Western blot assay to test the expression of the heterologous proteins. (FIG. 5B) Quantification of the expression by densitometry analysis (ImageJ software). Sample codes: 1: CV8_HA_c2 (R49.4). 2:CV8_HA_c2_D (R68.4). 3: CV8_D (R75.4). 4: CV8_DL (R76.4). 5: CV8_HA_c2_DL (R77.4), 6:CV8.

FIGS. 6A-6C. Histogram showing the activity of engineered strains on S. aureus, PAO1 and SAT290 biofilms formed in vitro. (FIGS. 6A-6C) The biofilms were treated with the supernatant of the indicated M. pneumoniae strains over an incubation period of 6 hours.

FIG. 7. Effect of M. pneumoniae strains on complex biofilms formed by S. aureus and P. aeruginosa. Crystal violet assay showing the effect of the indicated strains on the biofilms formed by S. aureus (left). P. aeruginosa SAT290 (middle), and a mix of S. aureus and SAT290 cells (10:1 ratio. right). Tested samples from left to right for each condition: HF (Hayflick medium), CV8, CV8_D (R75.4), CV8_DL (R76.4), CV8_HA_c2 (R49.4), CV8_HA_c2_D (R68.4), CV8_HA-c2_DL (R77.4). The arrows indicate the best result.

FIGS. 8A and 8B. Biofilm dispersion of the indicated P. aeruginosa clinical isolates were exposed to the supernatant of the indicated M. pneumoniae strains, with or without antibiotics. (FIG. 8A) Crystal violet assay showing that the antibiotic Ceftazidime-Avibactam becomes active on the biofilm of the clinical isolate SAT290 when combined to CV8_HA. (FIG. 8B) Similar results of panel A, using the clinical isolate C117 and the antibiotics Piperacillin-Tazobactam.

FIGS. 9A and 9B. Evaluation of the ratio of CFUs by genetically engineered M. pneumoniae strains expressing only a subset of the envisaged therapeutic proteins. Growth rates were monitored for the CV8_DL strain and the CV8_HA strain which were either cultured in separate HF growth plates (black circles) or co-cultured in a single HF growth plate (black squares). (FIG. 9A) Direct comparison between the CFU ratios of the two growth regimens. (FIG. 9B) Correlation of the CFU ratios observed for both growth regimens. CFU=Colony Forming Unit.

FIG. 10. Expression of heterologous proteins by different M. pneumoniae strains engineered in different sequential orders. Western blot analysis of the indicated strains. The strain WT_HA+D was originated by first transforming a WT strains with a transposon carrying the genes for PelAh, PslGh and AI-II′ (transposon HA), and then with a transposon carrying the Dispersin B gene. The strains WT_D+HA was originated with the opposite order of transposons.

FIG. 11. Results of PET assay in mice models of S. aureus biofilms formed in catheters in vivo. C179 are animals treated with empty chassis PB_Ch3 (when left untreated, the biofilm increases over time). Upon treatment with CV2DBlys, R2.5 (PB_Ch3_AI-II′_PelAh_Pslh_DispB) or R3.5 (PB_Ch3_AI-II′_PelAh_Pslh_DispB_lyso) strains, a near identical treatment effect is observed.

FIG. 12. Comparison of the efficacy of different engineered M. pneumoniae strains on in vitro biofilms formed by S. aureus, P. aeruginosa (PAO1), or complex biofilms (S. aureus+PAO1). From left to right for each biofilm: Hayflick medium, PBCH3, CV8_Disp, PBCh3_HA, and PBCh3_HA-Disp.

FIG. 13. Efficacy of resulting engineered M. pneumoniae strain against in vivo biofilms. Left panel: efficacy against a biofilm formed by S. aureus in vivo on a catheter measured by positron tomography with [¹⁸F]-FDG-MicroPET. Plots showing the SUV60 (standardized uptake value) variation (%) between day 1 and day 4 of the different treatments. Right panel: efficacy against a biofilm formed by P. aeruginosa PAO1 in vivo on a catheter measured by the number of colony forming units (CFU). Each circle represents an individual animal, whereas mean±SD is represented with lines inside each group.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

The term “subject”, “patient”, and “subject in need” may be used interchangeably herein and refer to animals, preferably warm-blooded animals, more preferably vertebrates, and even more preferably mammals specifically including humans and non-human mammals. The term “mammals”, or “mammalian subjects” refers to any animal classified as such and hence include, but are not limited to humans, domestic animals, commercial animals, farm animals, zoo animals, sport animals, pet and experimental animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Preferred patients are human subjects. Particularly preferred are human subjects, including both genders and all age categories thereof.

Amino acids are referred to herein with their full name, their three-letter abbreviation or their one letter abbreviation. Unless explicitly stated otherwise, reference herein to any peptide, polypeptide, protein, or nucleic acid, or fragment thereof may generally also encompass modified forms of said peptide, polypeptide, protein, or nucleic acid, or fragment thereof, such as bearing post-expression modifications including the following non-limiting examples: phosphorylation, glycosylation, lipidation, methylation, cysteinylation, sulphonation, glutathionylation, acetylation, oxidation of methionine to methionine sulphoxide or methionine sulphone, combinations thereof.

A first aspect of the invention is related to a genetically modified Mycoplasma bacterium comprising in its genome a nucleotide arrangement wherein said oligonucleotide arrangement comprises: i) a first nucleotide sequence encoding a first heterologous exopolysaccharide hydrolyzing enzyme under the control of a promoter or a functional variant of said promoter or fragment thereof which is active in said Mycoplasma bacterium, and ii) at least one further nucleotide sequence encoding further heterologous exopolysaccharide hydrolyzing enzymes under the control of a promoter or a functional variant of said promoter or fragment thereof which is active in said Mycoplasma bacterium. Preferably, the first exopolysaccharide hydrolyzing enzyme is Dispersin B. In alternative preferred embodiments, the one or more further exopolysaccharide hydrolyzing enzymes are selected from the group consisting of: Alginate lyase AI-II′, PelAh, PslGh, or any combination thereof. In particularly preferred embodiments, the first exopolysaccharide hydrolyzing enzyme encoded by the first nucleotide sequence comprises, or consists of Dispersin B, and the second nucleotide sequence comprises, or consists, both encoded alginate lyase AI-II′, PelAh, and PslGh. Dispersin B is active against biofilms formed by Staphylococcus aureus whereas PelAh, PslGh and Alginase AI-II′ are active against biofilms formed by Pseudomonas aeruginosa. In certain embodiments, the first and second nucleotide sequence are located adjacent to each other in the genomic sequence of the Mycoplasma bacterium. However, equally envisaged is the prevalence of the first and sequence nucleotide sequence in distinct genomic locations of the Mycoplasma bacterium. Indeed, according to the present state of the technical field, a skilled person appreciates that predominantly the occurrence and expression of the at least two heterologous exopolysaccharide hydrolyzing enzymes will be crucial for achieving the stated and/or implied technical effects mentioned throughout the present disclosure, and that the precise genomic location, either considered in isolation or relative to each other, is of secondary importance in the context of the present invention.

In certain embodiments, the Dispersin B referenced to herein has at least 65%, preferably 75%, more preferably 85%, yet more preferably at least 95%, most preferably 100% sequence identity to SEQ ID NO: 19 reproduced below:

MNYIKKIILSLFLLGLFSVLNCCVKGNSIYPQKTSTKQTGLMLDI
ARHFYSPEVIKSFIDTISLSGGNFLHLHFSDHENYAIESHLLNQR
AENAVQGKDGIYINPYTGKPFLSYRQLDDIKAYAKAKGIELIPEL
DSPNHMTAIFKLVQKDRGVKYLQGLKSRQVDDEIDITNADSITFM
QSLMSEVIDIFGDTSQHFHIGGDEFGYSVESNHEFITYANKLSYF
LEKKGLKTRMWNDGLIKNTFEQINPNIEITYWSYDGDTQDKNEAA
ERRDMRVSLPELLAKGFTVLNYNSYYLYIVPKASPTFSQDAAFAA
KDVIKNWDLGVWDGRNTKNRVQNTHEIAGAALSIWGEDAKALKDE
TIQKNTKSLLEAVIHKTNGDE

In certain embodiments, the alginate lyase AI-II′ referenced to herein has at least 65%, preferably 75%, more preferably 85%, yet more preferably at least 95%, most preferably 100% sequence identity to SEQ ID NO: 20 reproduced below:

MPAAAPGKNFDLSHWKLQLPDANTTEISSANLGLGYTSQYFYTDT
DGAMTFWAPTTGGTTANSSYPRSELREMLDPSNSKVNWGWQGTHT
MKLSGKTVQLPSSGKIIVAQIHGIMDDGTNAPPLVKAVFQDGQLD
MQVKQNSDGTGSDVHNYFTGIKLGDLYNMEIRVTDGVAYVTMNGD
TRSVDFVGKDAGWKNLKYYFKAGNYVQDNTSTGGSAIAKLYSLSV
SHSN

In certain embodiments, the PelAh referenced to herein has at least 65%, preferably 75%, more preferably 85%, yet more preferably at least 95%, most preferably 100% sequence identity to SEQ ID NO: 21 reproduced below:

MGGPSSVAFWYAERPPLAELSQFDWVVLEAAHLKPADVGYLKEQG
STPFAYLSVGEFDGDAAAIADSGLARGKSAVRNQAWNSQVMDLAA
PSWRAHLLKRAAELRKQGYAGLFLDTLDSFQLQAEERREGQRRAL
ASFLAQLHRQEPGLKLFFNRGFEVLPELPGVASAVAVESIHAGWD
AAAGQYREVPQDDRDWLKGHLDALRAQGMPIVAIDYLPPERRDEA
RALAARLRSEGYVPFVSTPALDYLGVSDVEVQP

In certain embodiments, the PslGh referenced to herein has at least 65%, preferably 75%, more preferably 85%, yet more preferably at least 95%, most preferably 100% sequence identity to SEQ ID NO: 22 reproduced below:

MEIQVLKAPRAVVWKDFLGVNAQFLWFSPERYNKQIDRLQDLGLE
WVRLDLHWDRLETAEDQYQLASLDQLVKDLEARQLKSVFYLVGSA
RFITTAPFYSPFQDQYPPRDPEVFARRMAMLSQRYPSVAAWQVWN
EPNLIGFWRPKADPEGYAKLLQASTIALRMVDPEKPVVSAGMAFF
SEMPDGRTMFDALGHLGVESLGTIATYHPYTQLPEGNYPWNLDFV
SHANQINRALRNAGVPAIWSTEWGWSAYKGPKELQDIIGVEGQAD
YVLRRLALMSALDYDRIFLFTLSDLDQRASVRDRDYGLLDLDANP
KPVYLALQRFLKVTGPKLRPADPPVTEDLPDGSFSIGWTREDGRN
VWLFWSARGGNVRLPKLKEATLHDPLSGKVTPLSGSDGLEVPVKS
SLQMLVWE

Previous work of the inventors focussed on the generation of genetically modified Mycoplasma bacteria that are able to disperse S. aureus biofilms or P. aeruginosa biofilms. These technological advantages were enabled by in-house developed genomic engineering technologies as described in detail in co-pending application PCT/EP2021/052110. While said bacteria provide numerous advantages over presently available S. aureus and P. aeruginosa biofilm treatment products and methods, certain drawbacks are still contained in said approach. For example, while two separate engineered Mycoplasma strains allow flexibility in treatment concentrations, this obliges the manufacturing of two separate strains, each adhering to demanding production (i.e. culturing) pipelines, both from a regulatory and compliance point of view. Furthermore, using two separate strains would inevitably mean that these strains would need to be brought together (i.e. mixed) to achieve efficient dispersion of a biofilm which in clinical relevant settings is de facto characterised by a heterogenous bacterial composition. Regardless of whether this is done prior to administration to the patient (i.e. storage in a single administration unit), or after administration to the patient of each strain separately, there is little to no control possible over how these two strains might react to each other. For example, when live Mycoplasma bacteria according to the invention are stored in a single administration unit, slight differences in e.g. growth rate or resilience against suboptimal storage conditions might lead to an overrepresentation of one strain, and hence diminishing or even eliminating the therapeutic properties of the other strain. The same reasoning applies mutatis mutandis to situations where the two strains are only brought together in the patient, where there is no guarantee that both strains will propagate equally efficient. Hence, producing a genetically modified Mycoplasma strain that is efficient in in vivo dispersal of clinical relevant biofilms such as those produced in the respiratory tract of a patient due to the co-occurrence of S. aureus and P. aeruginosa would be beneficial for both the patient (safety, control) and a manufacture (lowered production cost).

“Mycoplasma”, “Mycoplasma bacteria”, or “Mycoplasmas” as used interchangeably herein refers to the mollicute genus Mycoplasma which is characterized by lack of a cell wall around their cell membranes. Therefore, the plasma membrane forms the outer boundary of the Mycoplasma bacterial cell. Due to the absence of a cell wall, Mycoplasma has been found to have versatile shapes ranging from round to oblong, and display pleomorphism. “Pleomorphism” as used herein is a term used in histology and cytopathology to describe cells and/or their nuclei that may contain variable sizes, shape and staining. Culturable Mycoplasma species typically form small umbonate colonies on agar. The exact shape of the Mycoplasmas may depend on numerous parameters including osmotic pressure, nutritional quality of the culture medium, and growth phase. Certain Mycoplasma bacteria may be filamentous in their early and exponential growth phases or when attached to surfaces or other cells. The filamentous form may be transitory, and in certain conditions the filaments may branch or fragment into chains of cocci or individual vegetative cells. Alternative species are typically coccoid and do not develop a filamentous phase. Certain species develop specialized attachment tip structures involved in the process of colonization and/or contribute to virulence. Mycoplasma bacteria comprise 16S and 70S type ribosomes and a replicating disc to assist the replication process, and isolation of the genetic material. Mycoplasma bacteria may either live as saprophytes or more commonly as parasites. The term “saprophytes” refers to the chemoheterotrophic extracellular digestion that takes place in the processing of decayed organic matter. Mycoplasma bacteria are commonly described as one of the smallest and simplest self-replicating organisms known to date. Naturally occurring Mycoplasma genomes vary from about 500kilobases (kb) to 1500 kb and GC contents between 23-41 mole percent (mol %) have been described.

Different Mycoplasma species have been described and catalogued in the art (inter alia in Thompson et al., Towards a genome based taxonomy of Mycoplasmas, 2011). It is evident to a skilled person that the term Mycoplasma additionally includes any Mycoplasma strain or species that is generated by genetic or chemical synthesis, or any sort of rational design and/or the reorganization of a naturally occurring Mycoplasma genomic sequence and that the term therefore also covers those Mycoplasma strains and species that are termed “synthetic Mycoplasma”, alternatively “Mycoplasma laboratorium”. “Mycoplasma synthia”, or even short “Synthia” in the art (Gibson et al., Creation of a bacterial cell controlled by a chemically synthesized genome, Science, 2010).

The term “active” as described herein indicates the capacity of the nucleotide encoded gene product to fulfill its commonly accepted function in the bacterium, or when reference is made toward activity of a bacterium as a whole, the capacity of the bacterium to fulfill its envisaged function (i.e., in the context of the present invention, a therapeutic function). Hence, for a gene product such as an enzyme to be active, it is understood that expression of the gene product is required in the bacterium. A skilled person understands that a heterologous gene product expressed by a bacterium does not equal that activity of a gene product can be observed upon expression of said heterologous gene product due to differences in for example post translational machinery present in different organisms. Assays to determine the activity of gene products, in particular to measure the enzymatic activity when said gene product is an enzyme, have been described in detail in the art (Burns et al., Methods for the Measurement of a Bacterial Enzyme Activity in Cell Lysates and Extracts, Biol Proced Online, 1998). A skilled person further understands that the “degree” or “level” of activity depends on numerous parameters, including but not limited to substrate availability, competing enzymes, and half-life of both substrate and enzyme. Hence, “active” as used herein can be interpreted as a quantitative reduction of substrate amount after incubation with an enzyme.

Methods and protocols to introduce oligonucleotide arrangements into bacteria, i.e. methods of bacterial transformation, are known to a person skilled in the art (Johnston et al., Bacterial transformation: distribution, shared mechanisms and divergent control, Nature reviews Microbiology, 2014). The term “transformation” is indicative for a genetic alteration of a cell resulting from the direct uptake and incorporation of exogenous genetic material. Transformation is a horizontal gene transfer process and is commonly used in context of introducing foreign DNA to a bacterial, yeast, plant, animal, or human cell. Cells capable of taking up foreign DNA are named competent cells. In other embodiments, transformation may be indicative for the insertion of new genetic material into animal and human cells, albeit the term “transfection” is more common for these cells. Related hereto, terms such as “heterologous” or “exogenous” which are ubiquitously used throughout the present disclosure indicate that a certain moiety (in the context of the present invention, a nucleotide sequence and/or the encoded protein) is not occurring in the natural, unmodified version of the genetically modified Mycoplasma bacterium, in the context of the present invention preferably a M. pneumoniae bacterium. Thus, in accordance with the generally accepted nomenclature in the fields of molecular and synthetic biology, “heterologous expression” indicates the expression of a gene or part of a gene in a host organism which does not naturally has this gene or gene fragment.

Non-limiting examples of suitable transformation methods that can be applied to bacteria include heat-shock transformation and electroporation. In heat shock transformation, artificial competence is typically induced by making the cell permeable to DNA by subjecting them to non-physiological conditions. In such a typical transformation experiment, the cells are incubated in a solution containing divalent cations often in cold conditions, before the cells are exposed to a heat shock. It is theorized that exposure of the cells to divalent cations are responsible for a weakening of the cell surface structure, rendering it (more) permeable to DNA. The heat shock generates a thermal imbalance across the membrane, forcing entry of DNA through cell pores (i.e. adhesion zones or Bayer junctions) or through the damaged cell wall. An alternative method to induce transformation is by means of electroporation. which is hypothesized to create pores in the cellular membrane. In electroporation the bacterial cells are briefly exposed to an electric field of 10-20 kV/cm. After the shock, cellular membrane repair mechanisms remove the pores.

The term “oligonucleotide arrangements” as used herein, or synonymously “nucleotide sequences”. “polynucleotide arrangements”, “polynucleotide sequences”, refers to a sequence of a multitude of nucleotides physically connected to form a nucleotide sequence. Unless the contrary is mentioned, the oligonucleotide arrangements are not presented in their naturally occurring genome. Means and methods to obtain, generate and modify isolated polynucleotide sequences are well known to a person skilled in the art (Alberts et al., Molecular Biology of the Cell. 4th edition, 2002).

In certain embodiments, the oligonucleotide arrangements described herein may be multiple DNA sequences.

In certain embodiments, an oligonucleotide arrangement as described herein is part of a bicistronic expression construct comprised in the genome of the genetically modified bacterium. In further embodiments, a oligonucleotide arrangement as described herein is comprised in a bacterial artificial chromosome, which is in the context of the present invention also considered to be part of the bacterial genome.

The term “promoter” as defined herein is a region of DNA that initiates transcription of a particular gene and hence enables a gene to be transcribed. A promoter is recognized by RNA polymerase, which then initiates transcription. Thus, a promoter contains a DNA sequence that is either bound directly by, or is involved in the recruitment, of RNA polymerase. A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene. Promoters may be located in close proximity of the start codon of genes, in preferred embodiments on the same strand and typically upstream (5′) of the gene. Promoters may vary in size, and are preferably from about 100 to 1000 nucleotides long. In certain embodiments, the promoter may be a constitutive promoter. A constitutive promoter is understood by a skilled person to be a promoter whose expression is constant under the standard culturing conditions, i.e. a promoter which expresses a gene product at a constant expression level. In alternative embodiments, the promoter may be an inducible (conditional) promoter. It is understood that inducible promoters are promoters which are responsive at least one induction cue. Inducible promoters, and more specifically bacterial inducible promoter systems have been described in great detail in the art (inter alia in Brautaset et al., Positively regulated bacterial expression systems, Microbial biotechnology, 2009). In certain embodiments, the inducible promoter is chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures). An inducible promoter can also be regulated by other transcription factors that are constitutive or are themselves directly regulated by chemical or physical cues. In certain embodiments, the promoter is a TetR promoter part of a Tet-On or Tet-off system (Krueger et al., Tetracycline derivatives: alternative effectors for Tet transregulators,

Biotechniques, 2004, and, Loew et al., Improved Tet-responsive promoters with minimized background expression, BioMedCentral Biotechnology, 2010). In further embodiments, the concatenation of different sequence elements may be considered as an operon. “Operon” as used herein refers to a functional unit of DNA containing a cluster of genes in which all genes are controlled by a single promotor. It is evident to a skilled person that genes from an operon are co-transcribed. Transcribed genes from an operon are transcribed to a single mRNA strand and may be either translated together in the cytoplasm or spliced to generate monocistronic mRNAs that may be translated separately. In certain embodiments, one or more oligonucleotide arrangements as described herein may comprise a regulatory sequence.

“Control sequences” or “regulatory sequences” as used interchangeably herein refer to any nucleotide sequence which capable of increasing or decreasing the expression of specific genes. This regulation may be imposed by either influencing transcription rates, translation rates, or by modification of the stability of the sequence. In further embodiments, the polynucleotide sequence comprises regulatory elements such as but not limited to the following: enhancers, selection markers, origins of replication, linker sequences, polyA sequences, terminator sequence, and degradation sequences. In certain embodiments, at least one oligonucleotide arrangement comprises one or more suitable control sequences. In certain embodiments, the control sequences are identical for all oligonucleotide arrangements. In alternative embodiments, different control sequences are used for or within different oligonucleotide arrangements. In certain embodiments, the control sequences are control sequences naturally occurring in Mycoplasma bacteria. In other embodiments, the control sequences are adapted to perform their intended function in Mycoplasma bacteria. It is evident to the skilled person that any component of the oligonucleotide arrangement as described herein may further comprise tag sequences that ameliorate purification or localization of either the nucleotide sequence, or one or more gene products encoded in the nucleotide sequences of the oligonucleotide arrangement. Both oligonucleotide motifs and sequences that bind to other oligonucleotides or proteins and amino acid motifs or sequences are envisaged.

The term “exopolysaccharide hydrolyzing enzyme” as used herein, is indicative for any enzyme that is capable of performing exopolysaccharide hydrolysis. As envisaged herein, the term enzyme includes biologically active analogs, (natural and synthetic) variants, fragments and chemically modified derivatives of the enzyme, which are capable of degrading exopolysaccharides. According to the present invention, the primary, secondary and/or tertiary structure of the enzyme can be modified as long as its biological activity is retained. “Exopolysaccharides” are a major component of the extracellular polymeric substance that establishes the functional and structural integrity of biofilms. are high-molecular-weight polymers that are composed of sugar residues and are secreted by a microorganism into the surrounding environment. In accordance with the common nomenclature in the technical field, the term “exopolysaccharide” refers to a component of the Extracellular Polymeric Substance (EPS), unless specifically mentioned otherwise. A skilled person is aware that EPSs comprise in addition to exopolysaccharides also DNA, lipid, and further organic substances (Flemming et al., Physico-Chemical Properties of Biofilms, Biofilms: Recent Advances in their Study and Control, 2000). Hence, when reference is made to “exopolysaccharide hydrolyzing enzyme” herein, said term excludes any enzyme that is not able to process the exopolysaccharides as such but is able to process non-exopolysaccharide components of the EPS. For example, a DNAse is not considered an exopolysaccharide hydrolyzing enzyme in the context of the present disclosure. Exopolysaccharides generally comprise monosaccharides and some non-carbohydrate substituents (including as acetate, pyruvate, succinate, and phosphate). Functions of exopolysaccharides have been described in detail in the art (Harimawan and Ting, Investigation of extracellular polymeric substances (EPS) properties of P. aeruginosa and B. subtilis and their role in bacterial adhesion. Colloids and Surfaces B: Biointerfaces. 2016). Non-limiting examples of suitable exopolysacchararide hydrolyzing enzymes are further discussed below. The term “biofilm” as used herein is a term that indicates any syntrophic consortium of microorganisms such as bacteria in which cells adhere to one another and/or to a surface. Biofilms are characterized by a viscous extracellular matrix composed of extracellular polymeric substances produced by the bacteria (Lopez. et al., Biofilms, 2010). Biofilms can form in natural, medical, and industrial settings. In medical settings, biofilm is a characteristic of several difficult to treat diseases including but not limited to cystic fibrosis and chronic obstructive pulmonary disease. Furthermore, biofilm formation on medical devices such as catheters and/or implants is responsible for an increasing incidence of chronic infections that are hard to effectively treat. Biofilms hamper treatment of such infection by different mechanisms, such as an increased amount of persister cells that are present in the biofilm which are non-dividing cells with a high antibiotic resistance (Lewis, Persister cells and the riddle of biofilm survival, Biochemistry. 2005). Besides persister cells, the biofilms also achieve increased protection from antibiotics by the extracellular matrix, which acts as a physical barrier. A skilled person is aware that specific molecular mechanisms and biofilm constituents vary between biofilms formed by different microorganisms. However, the extracellular matrix described above (comprising exopolysaccharides, proteins, and/or DNA) is a general feature of biofilms (Monds and O'Toole. The developmental model of microbial biofilms: ten years of a paradigm up for review. Trends Microbiol, 2009) The nature of the matrix containing exopolysaccharides is dependent on numerous parameters such as but not limited to the involved microorganisms, growth conditions, medium, and substrates. These parameters have been described in the art and are therefore known to a skilled person (Branda et al., Biofilms: the matrix revisited. Trends Microbiol, 2005).

In a further aspect, the present invention provides a genetically modified Mycoplasma bacterium comprising in its genome a deletion, substitution, and/or insertion of one or more nucleotides in the operons of the Ca2+ dependent cytotoxic nuclease gene (MPN133) or orthologues thereof and ADP-ribosyltransferase CARDS gene (MPN372) or orthologues thereof, that reduce the pathogenicity and/or immunogenicity of said Mycoplasma bacterium compared to a reference M129-B7 Mycoplasma pneumoniae bacterium, said reduction in pathogenicity and/or immunogenicity being characterized by a reduction of toxicity by at least 30% upon introduction into a host organism when compared to reference M129-B7 Mycoplasma pneumoniae bacterium, wherein said Mycoplasma bacterium further comprises in its genome an oligonucleotide arrangement, said oligonucleotide arrangement comprising: a nucleotide arrangement wherein said oligonucleotide arrangement comprises: i) a first nucleotide sequence encoding a first heterologous exopolysaccharide hydrolyzing enzyme under the control of a promoter or a functional variant of said promoter or fragment thereof which is active in said Mycoplasma bacterium, and ii) at least one further nucleotide sequence encoding further heterologous exopolysaccharide hydrolyzing enzymes under the control of a promoter or a functional variant of said promoter or fragment thereof which is active in said Mycoplasma bacterium. Preferably, the first exopolysaccharide hydrolyzing enzyme is Dispersin B. In alternative preferred embodiments, the one or more further exopolysaccharide hydrolyzing enzymes are selected from the group consisting of: Alginate lyase AI-II′, PelAh, PslGh, or any combination thereof. In particularly preferred embodiments, the first exopolysaccharide hydrolyzing enzyme encoded by the first nucleotide sequence comprises, or consists of Dispersin B, and the second nucleotide sequence comprises, or consists, both encoded alginate lyase AI-II′, PelAh, and PslGh.

In a highly preferred embodiment of any of the foregoing aspects, the genetically modified Mycoplasma bacterium further comprises one or more antimicrobial agents not encoded by or expressed by a Mycoplasma pneumoniae bacterium, preferably wherein the one or more antimicrobial agents are not encoded by or expressed by the reference M129-B7 (ATCC identifier 29342) Mycoplasma pneumoniae bacterium (i.e. a heterologous antimicrobial agent). In further preferred embodiments, the one or more antimicrobial agents are comprised in the Mycoplasma bacterium genome. In further preferred embodiments, the one or more antimicrobial agents are encoded in a nucleotide sequence comprised in the Mycoplasma bacterium genome under the control of a naturally occurring Mycoplasma promoter. In alternative further preferred embodiments, the one or more antimicrobial agents are encoded in a nucleotide sequence comprised in the Mycoplasma bacterium genome under the control of a naturally occurring Mycoplasma promoter.

In an alternative aspect to the above, the invention provides in a pharmaceutical dosage unit comprising as a first component:
a genetically modified Mycoplasma bacterium comprising in its genome a nucleotide arrangement wherein said oligonucleotide arrangement comprises: i) a first nucleotide sequence encoding a first heterologous exopolysaccharide hydrolyzing enzyme under the control of a promoter or a functional variant of said promoter or fragment thereof which is active in said Mycoplasma bacterium, and ii) at least one further nucleotide sequence encoding further heterologous exopolysaccharide hydrolyzing enzymes under the control of a promoter or a functional variant of said promoter or fragment thereof which is active in said Mycoplasma bacterium;
   and as a second component:
one or more antimicrobial agents not encoded by or expressed in a reference Mycoplasma pneumoniae bacterium (i.e. one or more heterologous antimicrobial agents), preferably wherein the reference Mycoplasma pneumoniae bacterium is reference M129-B7 Mycoplasma pneumoniae strain (ATCC identifier 29342).

Preferably, the first exopolysaccharide hydrolyzing enzyme is Dispersin B. In alternative preferred embodiments, the one or more further exopolysaccharide hydrolyzing enzymes are selected from the group consisting of: Alginate lyase AI-II′, PelAh, PslGh, or any combination thereof. In particularly preferred embodiments, the first exopolysaccharide hydrolyzing enzyme encoded by the first nucleotide sequence comprises, or consists of Dispersin B, and the second nucleotide sequence comprises, or consists, both encoded alginate lyase AI-II′, PelAh, and PslGh.

In a further embodiment, the genetically modified bacterium comprised in the pharmaceutical dosage unit as first component further comprises in its genome a deletion, substitution, and/or insertion of one or more nucleotides in the operons of the Ca2+ dependent cytotoxic nuclease gene (MPN133) or orthologues thereof and ADP-ribosyltransferase CARDS gene (MPN372) or orthologues thereof, that reduce the pathogenicity and/or immunogenicity of said Mycoplasma bacterium compared to a reference M129-B7 (ATCC identifier 29342) Mycoplasma pneumoniae bacterium, said reduction in pathogenicity and/or immunogenicity being characterized by a reduction of toxicity by at least 30% upon introduction into a host organism when compared to reference M129-B7 Mycoplasma pneumoniae bacterium.

In certain embodiments, the genetically modified Mycoplasma bacterium as described herein, comprises a functional modification such as a deletion, substitution, and/or insertion in one or more genes or operons encoding a protein capable of eliciting Guillain-Barre in a host organism, preferably in MPN257and/or MPN483. Hence, in certain embodiments, the genetically modified Mycoplasma bacterium as described herein comprises a function modification such as a deletion, substitution, and/or insertion in one or more genes or operons of MPN257, MPN483, MPN133, MPN372, or any combination thereof. In further embodiments, the genetically modified Mycoplasma bacterium as described herein comprises a function modification such as a deletion, substitution, and/or insertion in one or more genes or operons of MPN257. MPN483, MPN133, MPN372, MPN051 or any combination thereof.

A person skilled in the art is aware that the terms “pharmaceutical dosage unit”. “pharmaceutical dosage form”, or short “dosage unit” or “dosage form” can be used interchangeably herein and are meant to describe one or more “pharmaceutical compositions”, “pharmaceutical formulations”, or “pharmaceutical preparations” containing a genetically modified Mycoplasma bacterium as active pharmaceutical ingredient, formulated with a pharmaceutically acceptable excipient, and manufactured or sold with the approval of a governmental regulatory agency as part of a therapeutic regimen for the treatment of disease in a mammal. It is evident that pharmaceutical dosage units are indicative for those dosage units that comprise a therapeutically effective amount of genetically modified Mycoplasma bacteria, or at least an amount of genetically modified Mycoplasma bacteria that, when introduced into a host organism as live bacteria, can propagate to express or deliver a therapeutically effective amount of a desired gene product and/or bacterial cargo.

The term “therapeutically effective amount” as used herein, refers to an amount of active compound or pharmaceutical agent that elicits the biological or medicinal response in a subject that is being sought by a researcher, veterinarian, medical doctor or other clinician, which may include a reduction or complete removal of the symptoms associated with the disease or condition being treated. Methods to determine pharmaceutically effective amounts are known in the art and are therefore known to a skilled person. It is further evident that therapeutic effective amounts are determined in function of the specific subject in need of treatment. Further, a wording such as “a subject in need of treatment” includes any subject or group of subjects that would benefit from treatment of a given condition. Such subjects may include, without limitation, those that have been diagnosed with a condition susceptible to treatment with the genetically modified Mycoplasma bacterium, those prone to develop said condition and/or those in who said condition is to be prevented.

In certain embodiments, the one or more nucleotide encoded exopolysaccharide hydrolyzing enzymes and the one or more nucleotide encoded heterologous antimicrobial proteins are each under the control of the same (i.e. identical) promoter or a functional variant of said promoter(s) or fragment thereof. In alternative embodiments, the one or more nucleotide encoded exopolysaccharide hydrolyzing enzymes and the one or more heterologous antimicrobial proteins are cach under the control of a distinct (i.e. different) promoter or a functional variant of said promoter(s) or fragment thereof. In certain embodiments where the third oligonucleotide arrangement encoding a heterologous DNA degrading enzyme and/or heterologous proteinase is present, the one or more nucleotide encoded exopolysaccharide hydrolyzing enzymes, the one or more nucleotide encoded antimicrobial proteins and/or the one or more nucleotide encoded DNA degrading enzymes are each under the control of the same (i.e. identical) promoter or a functional variant of said promoter or fragment thereof. In alternative embodiments where the third oligonucleotide arrangement encoding a DNA degrading enzyme and/or proteinase is present, the one or more nucleotide encoded exopolysaccharide hydrolyzing enzymes, the one or more antimicrobial proteins and/or the one or more DNA degrading enzymes are each under the control of a distinct (i.e. different) promoter or a functional variant of said promoters or fragment thereof.

In certain embodiments, at least one of the nucleotide encoded heterologous exopolysaccharide hydrolyzing enzymes is operably linked to be under the control of a single promoter or a functional variant of said promoter or fragment thereof. In certain embodiments, a least two of the nucleotide encoded heterologous exopolysaccharide hydrolyzing enzymes is operably linked to be under the control of a single promoter or a functional variant of said promoter or fragment thereof. The wording “operably linked” refers to a multitude of genetic elements that are joined as part of the same nucleic acid molecule, suitably positioned and oriented for transcription to be initiated from the promoter. DNA operably linked to a promoter is under transcriptional initiation regulation of the promoter or in functional combination therewith. In certain embodiments, the at least one of the nucleotide encoded heterologous exopolysaccharide hydrolyzing enzymes, at least one of the nucleotide encoded heterologous antimicrobial proteins, and/or at least one of the nucleotide encoded heterologous DNA degrading enzyme are comprised in a polycistronic construct, preferably bicistronic or tricistronic. The terms “polycistronic”, “bicistronic”, and “tricistronic” as used herein indicate that respectively multiple, two, or three separate proteins are encoded in a single messenger RNA. In certain embodiments, the polycistronic construct comprises one or more 2A peptides as described in the art (Liu et al., Systematic comparison of 2A peptides for cloning multi-genes in a polycistronic vector, Scientific Reports, 2017) that separates the at least one of the nucleotide encoded exopolysaccharide hydrolyzing enzymes, at least one of the nucleotide encoded antimicrobial proteins, and/or at least one of the nucleotide encoded DNA degrading enzyme. In further embodiments, the 2A peptide encoded in the polycistronic construct is selected from the group of 2A peptides consisting of T2A, P2A, E2A or F2A. In alternative embodiments, the at least one of the nucleotide encoded exopolysaccharide hydrolyzing enzymes, at least one of the nucleotide encoded antimicrobial proteins, and/or at least one of the nucleotide encoded DNA degrading enzyme are separated by one or more Internal Ribosomal Entry Site (IRES) sequences. IRES sequences and their use in bacterial systems have been described in the art and are therefore known to a skilled artisan (Colussi et al., Initiation of translation in bacteria by a structured eukaryotic IRES RNA, Nature, 2015).

The term “antimicrobial agent” as used herein refers to any agent, molecule, or substance which is capable of stopping the growth of microorganism or killing said microorganisms. Thus, the term encompasses both microbicides and bacteriostatic agents. The term further compasses different classes of molecules and substances, including but not limited to antibiotics, antimicrobial proteins (bacteriocins), defensins, and antimicrobial peptides which each are equally preferred antimicrobial agents in the context of the present invention. “Bacteriocins” as described herein, and interchangeably annotated in the art as “antimicrobial proteins” or “antimicrobial peptides” indicate proteogenic molecules that that demonstrate a toxic effect to (one or more classes of) bacteria. Antimicrobial proteins and peptides have been demonstrated to kill Gram negative and Gram positive bacteria. Unlike the majority of conventional antibiotics antimicrobial peptides frequently destabilize biological membranes, can form transmembrane channels, and may also have the ability to enhance immunity by functioning as immunomodulators. Both naturally occurring antimicrobial proteins (peptides) and synthetic antimicrobial peptides are envisaged herein. A large number of antimicrobial peptides have been described in the art, and are therefore known to a skilled person, as are their (potential) application(s) (for example in Reddy et al., Antimicrobial peptides: premises and promises, International Journal of Antimicrobial Agents, 2004). Non-limiting examples of suitable antimicrobial agents are further discussed below.

Different classes of bacteriocins have been described (e.g. in Soltani et al., Bacteriocins as a new generation of antimicrobial: toxicity aspects and regulations, FEMS Microbiology Reviews, 2020), and include without limitation gram-negative bacteriocins and gram-positive bacteriocins. In certain embodiments, the bacteriocin is a gram negative bacteriocin, optionally selected from the group consisting of microcins, colicin-like bacteriocins, and tailocins. Non-limiting examples of microcins include microcin V (E. coli), and Subtilosin A (B. subtilis). In alternative embodiments, the bacteriocin is a gram positive bacteriocin, optionally selected from the group consisting of: class I bacteriocins (small peptide inhibitors), class II bacteriocins (small heat-stable proteins <10 kDa), class III bacteriocins (heat-labile proteins >10 kDa), and class IV bacteriocins (complex bacteriocins containing lipid or carbohydrate moicties). A skilled person is well aware of certain databases to retrieve bacteriocins which include the BAGEL and BACTIBASE databases (respectively described in De Jong et al., BAGEL: A web-based bacteriocin genome mining tool, Nucleic Acids Research, 2006; and Hammami et al., BACTIBASE: a new web-accessible database for bacteriocin characterization. BMC Microbiology, 2007.

In any of the above aspects and embodiments describing the genetically modified Mycoplasma bacterium or pharmaceutical composition comprising said bacterium, the bacteriocin is preferably selected from the group consisting of: acidocin, actagardine, agrocin, alveicin, aureocin, aureocin A53, aureocin A70, bisin, carnocin, carnocyclin, caseicin, cerein, circularin A, colicin, curvaticin, divercin, duramycin, enterocin, enterolysin, epidermin, gallidermin, erwiniocin, gardimycin, gassericin A, glycinecin, halocin, haloduracin, klebicin, lactocin S, lactococcin, lacticin, leucoccin, lysostaphin, macedocin, mersacidin, mesentericin, microbisporicin, microcin, microcin S, mutacin, nisin, pacnibacillin, planosporicin, pediocin, pentocin, plantaricin, pneumocyclicin, pyocin, reutericin 6, reutericyclin, reuterin, sakacin, salivaricin, sublancin, subtilin, sulfolobicin, tasmancin, thuricin 17, trifolitoxin, variacin, vibriocin, warnericin, warnerin, or any combination thereof.

In a particularly preferred embodiment, the bacteriocin is lysostaphin. “Lysostaphin” as referenced to in the present disclosure refers to a 27 kDa glycylglycine endopeptidase expressed by Staphylococcus simulans. Lysostaphin acts by cleaving the crosslinking pentaglycine bridges in the peptidoglycan cell wall of Staphylococci species (Kun et al., Lysostaphin Cream Eradicates Staphylococcus aureus Nasal Colonization in a Cotton Rat Model, Antimicrobial agents and Chemotherapy, 2003). Lysostaphin (Uniprot Identifier P10547) is characterized by the following sequence (SEQ ID NO: 1):

MKKTKNNYYTRPLAIGLSTFALASIVYGGIQNETHASEKSNMDVS
KKVAEVETSKAPVENTAEVETSKAPVENTAEVETSKAPVENTAEV
ETSKAPVENTAEVETSKAPVENTAEVETSKAPVENTAEVETSKAP
VENTAEVETSKAPVENTAEVETSKAPVENTAEVETSKAPVENTAE
VETSKAPVENTAEVETSKAPVENTAEVETSKAPVENTAEVETSKA
PVENTAEVETSKALVQNRTALRAATHEHSAQWLNNYKKGYGYGPY
PLGINGGMHYGVDFFMNIGTPVKAISSGKIVEAGWSNYGGGNQIG
LIENDGVHRQWYMHLSKYNVKVGDYVKAGQIIGWSGSTGYSTAPH
LHFQRMVNSFSNSTAQDPMPFLKSAGYGKAGGTVTPTPNTGWKTN
KYGTLYKSESASFTPNTDIITRTTGPFRSMPQSGVLKAGQTIHYD
EVMKQDGHVWVGYTGNSGQRIYLPVRTWNKSTNTLGVLWGTIK

Similarly, in alternative embodiments, the bacteriocin comprised in the genetically modified Mycoplasma bacterium or comprising in the pharmaceutical dosage unit is characterized by a sequence identity of at least 65% to SEQ ID NO: 1, preferably at least 75% sequence identity to SEQ ID NO: 1, more preferably at least 75% sequence identity to SEQ ID NO: 1, most preferably at least 75% sequence identity to SEQ ID NO: 1.

In a yet more preferred embodiment. the bacteriocin is a truncated lysostaphin protein corresponding to SEQ ID NO: 1 wherein the endogenous signal sequence and pro-peptide sequence are removed and the truncated lysostaphin is therefore characterized by the following sequence (SEQ ID NO: 11):

MAATHEHSAQWLNNYKKGYGYGPYPLGINGGMHYGVDFFMNIGTP
VKAISSGKIVEAGWSNYGGGNQIGLIENDGVHRQWYMHLSKYNVK
VGDYVKAGQUIGWSGSTGYSTAPHLHFQRMVNSFSNSTAQDPMPF
LKSAGYGKAGGTVTPTPNTGWKTNKYGTLYKSESASFTPNTDIIT
RTTGPFRSMPQSGVLKAGQTIHYDEVMKQDGHVWVGYTGNSGQRI
YLPVRTWNKSTNTLGVLWGTIK

Similarly, in alternative embodiments, the bacteriocin comprised in the genetically modified Mycoplasma bacterium or comprising in the pharmaceutical dosage unit is characterized by a sequence identity of at least 65% to SEQ ID NO: 11. preferably at least 75% sequence identity to SEQ ID NO: 11, more preferably at least 75% sequence identity to SEQ ID NO: 11, most preferably at least 75% sequence identity to SEQ ID NO: 11.

In highly preferred embodiments of the invention the expression of the antimicrobial agent, which preferably is a lysostaphin, is controlled by a promoter sequence comprising a nucleotide sequence of at least 65% identity, preferably at least 75% identity, more preferably at least 85% identity, even more preferably at least 95% identity to the nucleotide sequence of SEQ ID NO: 6, most preferably wherein expression of lysostaphin is controlled by a promoter sequence having 100% sequence identity to SEQ ID NO: 6.

In yet alternative embodiments, the antimicrobial agent is an antimicrobial peptide. Antimicrobial peptides have been described extensively in the art on numerous occasions (e.g. in Huan et al., Antimicrobial Peptides: Classification, Design, Application and Research Progress in Multiple Fields,

Frontiers in Microbiology, 2020). In further embodiments, the antimicrobial peptide is a membranolytic antimicrobial peptide, an non-membranolytic peptide, or any combination of a membranolytic peptide and a non-membranolytic peptide. Thus, in certain embodiments, the antimicrobial peptide is selected from the group consisting of: Bacitracin, Dalbavanein, Daptomycin, Oritavancin, Teicoplanin, Telavancin, Vancomycin, Guavanin 2, Cecropin A, or any combination thereof. In yet further embodiments, the antimicrobial peptide is a concatenation of at least two antimicrobial peptides, wherein optionally the distinct antimicrobial peptides are separated from each other by a peptide linker sequence.

In certain embodiments, the bacteriocin comprised in the pharmaceutical dosage unit is an antibiotic. In further embodiments, the antibiotic is a bacterial membrane targeting antibiotic. In preferred embodiments, the antibiotic is selected from the group consisting of: Piperacillin, Tazobactam, Ciprofloxacin. Levofloxacin. Meropenem. Imipenem, Cilastatin. Amikacin. Ceftazidime, Avibactam. Ceftolozane, Ceftriaxone, Vancomycin, Linezolid, or any combination thereof. In further embodiments wherein the pharmaceutical dosage unit comprises as antimicrobial agent an antibiotic, said antibiotic is selected from the group comprising Piperacillin, Tazobactam, Meropenem, Imipenem, Cilastatin, and Vactomycin In a preferred embodiment, the antibiotic is Meropenem. In further embodiments, the Meropenem is present in an amount corresponding to a final concentration of about 100 μg/ml. In certain embodiments, the pharmaceutical dosage unit described herein comprises Piperacillin and Tazobactam, preferably wherein the Piperacillin-Tazobactam is present in an amount corresponding to a final concentration of about 500 μg/ml. In alternative embodiments, the pharmaceutical dosage unit as described herein comprises Imipenem and Cilastatin, preferably wherein the Imipenem-Cilastatin combination is present in an amount corresponding to a final concentration of about 300 μg/ml. In alternative embodiments, the pharmaceutical dosage unit comprises Vactomycin, preferably wherein the Vactomycin is present in an amount corresponding to a final concentration of about 200 μg/ml. Some of the above mentioned combinations are particularly preferred combinations in the context of the present invention. An exemplary combination hereof is the combination of Piperacillin and Tazobactam, wherein Tazobactam will prevent Piperacillin degradation. A further particularly preferred combination is the combination of Imipenen and Cilastatin, wherein Cilistatin will prevent Imipenen degradation. Yet a further particularly preferred combination is the combination of Ceftazidime and Avibactam, wherein Avibactam will prevent Ceftazidime degradation. Yet a further particularly preferred combination is the combination of Ceftolozane and Tazobactam, wherein Tazobactam will prevent Ceftozolane degradation.

In yet alternative embodiments, the antimicrobial agent is a defensin. The term “defensin” as used herein is to be interpreted according to its accepted meaning in the art (e.g. in Hazlett and Wu, Defensins in innate immunity, Cell and Tissue Research, 2011) and therefore refers to a group of small cysteine-rich cationic proteins that are able to act as host defense peptide, including defensin-like peptides. Synonyms known to a skilled person include but are not limited to “cationic antimicrobial proteins”, “neutrophil peptides”, and “gamma thionins”. Without limitation, the majority of defensin sequences are characterized by a length of from about 18 to about 45 amino acids which include a number of disulfide bonds, preferably about 3 or 4 disulfide bonds. Defensins are known to form small and compact folded structures typically characterized by a high positive charge. In addition, defensins are highly stable duc to the multiple disulfide bonds. In preferred embodiments, the defensin is a trans-defensin, cis-defensin, or a defensin-like protein. In further preferred embodiments, the defensin is a human defensin (i.e. a defensin which is naturally expressed in homo sapiens). In certain embodiments, the defensin is an α-defensin. β-defensin, or θ-defensin. In further embodiments, the defensin is a human α-defensin, human β-defensin, or human θ-defensin. In yet further embodiments, the defensin is a defensin selected from the group of defensins selected from the group of defensins selected from the group consisting of: neutrophil defensin 1, defensin alpha 1, neutrophil defensin 3, neutrophil defensin 4, defensin-5,defensin-6, defensin beta 1, defensin beta 4A, defensin beta 4B, defensin beta 103A, defensin beta 103B, defensin beta 104A, defensin beta 104B, defensin beta 105A, defensin beta 105B, defensin beta 106A, defensin beta 106B, defensin beta 107A, defensin beta 107B, defensin beta 108A, defensin beta 108B, defensin beta 108C, defensin beta 108D, defensin beta 108E, defensin beta 108F, defensin beta 109A, defensin beta 109B, defensin beta 109C, defensin beta 109D, defensin beta 109E, defensin beta 109F, defensin beta 110, defensin beta 112, defensin beta 113, defensin beta 114, defensin beta 115, defensin beta 116, defensin beta 117, defensin beta 118, defensin beta 119, defensin beta 121, defensin beta 122,defensin beta 123, defensin beta 124, defensin beta 125, defensin beta 126, defensin beta 127, defensin beta 128, defensin beta 129, defensin beta 130A, defensin beta 130B, defensin beta 130C, defensin beta 130D, defensin beta 131A, defensin beta 131B, defensin beta 131C, defensin beta 131D, defensin beta 131E, defensin beta 132, defensin beta 133, defensin beta 134, defensin beta 135, defensin beta 136,defensin-like proteins, or any combination thereof.

In certain embodiments, at least one oligonucleotide sequence as described herein comprises a constitutive promoter. In further embodiments, the constitutive promoter is a promoter having a sequence identity of at least 65%, preferably at least 75%, at least 80%, at least 85%, at least 90% to a promoter selected from the group consisting of P438 (SEQ ID NO: 2), EfTu (SEQ ID NO: 3), P1 (SEQ ID NO: 4), P2 (SEQ ID NO: 5), P3 (SEQ ID NO: 6), P4 (SEQ ID NO: 7), P5 (SEQ ID NO: 8), and Psyn (SEQ ID NO: 9). In yet further embodiments, the constitutive promoter is selected from the group consisting of P438 (SEQ ID NO: 2), EfTu (SEQ ID NO: 3), P1 (SEQ ID NO: 4), P2 (SEQ ID NO: 5), P3 (SEQ ID NO: 6), P4 (SEQ ID NO: 7), P5 (SEQ ID NO: 8), and Psyn (SEQ ID NO: 9). In certain embodiments, at least one oligonucleotide arrangement as described herein comprises a synthetic promoter with a nucleotide sequence of at least 65% identity, preferably at least 75% identity, more preferably at least 85% identity, most preferably at least 95% identity to the nucleotide sequence of SEQ ID NO: 3 (EfTu). The above-recited sequences are the following:

P438 promoter (SEQ ID NO 2)
TAGTATTTAGAATTAATAAAGT
EfTu promoter (SEQ ID NO 3):
GAAGACCTTTTGTGCTAACGCCAGTTTGGCAAATCAAGTTCTGAT
TTTGCAATTATTTTGCTCCATATGAATTACACTACTCCAAGAATT
ATAAGCCTCTCTACAGCTTTATCTCAAACTTATGTAAAATTAGAG
ACGTAATTCAAACAC
P1 promoter (SEQ ID NO 4):
TTCCCAAATTTTCGAAATAATATTGTATAATTAAAAATA
P2 promoter (SEQ ID NO 5):
TATTGCATAATATTGCATAATATTGTATAATTAAAAATA
P3 promoter (SEQ ID NO 6):
TATTGCATAATATTGTATAATATTGTATAATTAAAAATA
P4 promoter (SEQ ID NO 7):
TATTGTATAATATTGTATAATATTGTATAATTAAAAATA
P5 promoter (SEQ ID NO 8):
TTCCCAAATTTTCGAAATAATATTGTATAATTAAAAATA
TATTGTATAATTAAAAATATTGTATAATTAAAAATA
Psyn promoter (SEQ ID NO 9):
GTTAACGATTAAGATCAAAAAGTGCCTGGTATCGTAAAT
AATATTGTATAATTAAAAAAGAAT

Methods and tools to verify sequence homology or sequence identity between different sequences of amino acids or nucleic acids are well known to a person skilled in the art and include non-limiting tools such as Protein BLAST, ClustalW2, SIM alignment tool, TranslatorX and T-COFFEE. The percentage of identity between two sequences may show minor variability depending on the algorithm choice and parameters. The term “sequence identity” refers to the relationship between sequences at the nucleotide (or amino acid) level. The expression “% identical” is determined by comparing optimally aligned sequences, e.g. two or more, over a comparison window wherein the portion of the sequence in the comparison window may comprise insertions or deletions as compared to the reference sequence for optimal alignment of the sequences. The reference sequence does not comprise insertions or deletions. A reference window is chosen and the “% identity” is then calculated by determining the number of nucleotides (or amino acids) that are identical between the sequences in the window, dividing the number of identical nucleotides (or amino acids) by the number of nucleotides (or amino acids) in the window and multiplying by 100. Unless indicated otherwise, the sequence identity is calculated over the whole length of the reference sequence.

In certain embodiments, any of the herein described nucleotide encoded gene products may comprise an exposure signal and/or a secretion signal. By the term nucleotide encoded gene product is intended any protein encoded by a oligonucleotide arrangement as described herein. In certain embodiments, the nucleotide sequence encoding the heterologous exopolysaccharide hydrolyzing enzyme and/or the nucleotide sequence encoding the heterologous antimicrobial protein further comprises an exposure signal sequence or a secretion signal sequence. In alternative embodiments, the nucleotide sequence encoding the heterologous DNA degrading enzyme and/or proteinase comprises an exposure signal sequence or a secretion signal sequence. The term “exposure signal sequence” as used herein is indicative for sequences encoding exposure signal peptides that targets the linked protein for exposure on the cell membrane. “Secretion signal sequence” as used herein refers to a sequence provoking or mediating secretion of a protein.

In certain embodiments, the secretion signal sequence is a naturally occurring sequence in Mycoplasma, preferably M. pneumoniae. In yet further embodiments, the secretion signal sequence is a Mycoplasma, preferably M. pneumoniae secretion signal sequence. In alternative embodiments, the secretion signal sequence is a not-naturally occurring Mycoplasma sequence. Mycoplasma secretion signals have been described in International patent application WO2016/135281 and are therefore known to a person skilled in the art. A skilled person furthermore understands that (mutagenized) exposure or secretion signals may be further mutagenized to improve exposure or secretion respectively of one or more nucleotide-encoded heterologous gene products described herein.

In certain embodiments, the nucleotide sequence encoding the heterologous exopolysaccharide hydrolyzing enzyme, and/or the nucleotide sequence encoding the heterologous antimicrobial protein, and/or the nucleotide sequence encoding the heterologous DNA degrading enzyme further comprises a nucleotide sequence encoding an exposure signal sequence or a secretion signal sequence. In further embodiments, the nucleotide sequence encoding the heterologous exopolysaccharide hydrolyzing enzyme, and/or the nucleotide sequence encoding the heterologous antimicrobial protein, and/or the nucleotide sequence encoding the heterologous DNA degrading enzyme further comprises a nucleotide sequence encoding a secretion signal sequence from MPN036 (SEQ ID NO: 12), MPN142 (SEQ ID NO: 13), MPN645 (SEQ ID NO: 14), MPN400 (SEQ ID NO: 15), MPN200 (SEQ ID NO: 16), MPN213(SEQ ID NO: 17), MPN489 (SEQ ID NO: 18). In yet further embodiments, the nucleotide sequence encoding the heterologous exopolysaccharide hydrolyzing enzyme, and/or the nucleotide sequence encoding the heterologous antimicrobial protein, and/or the nucleotide sequence encoding the heterologous DNA degrading enzyme further comprises a nucleotide sequence encoding the optimized secretion signal from MPN142 (nucleotide sequence: SEQ ID NO: 23; amino acid sequence: SEQ ID NO: 10).

In certain embodiments, the exopolysaccharide hydrolyzing enzyme is a peptidoglycan hydrolase or a glycoside hydrolase. Both peptidoglycan hydrolases and glycoside hydrolases have been described in the art (Sharma et al., Prediction of peptidoglycan hydrolases-a new class of antibacterial proteins, BMC genomics, 2016, and Bourne et al., Glycoside hydrolases and glycosyltransferases: families and functional modules, Current opinion in structural biology, 2001).

Hence, the commonality between any of the aspects described herein is the genetically modified Mycoplasma bacterium comprising at least one nucleotide arrangement comprising at least two nucleotide sequences each encoding at least one exopolysaccharide hydrolyzing enzyme wherein one exopolysaccharide hydrolyzing enzyme is active against (i.e. lethal for, actively inhibiting propagation of, inhibiting the effects of, killing of) S. aureus and at least one exopolysaccharide hydrolyzing enzyme is active against P. aeruginosa, hence effectively enabling the ability of the genetically modified Mycoplasma bacterium to disperse biofilms having a composition being the result of co-occurrence of S. aureus and P. aeruginosa. Therefore, in certain embodiments, the exopolysaccharide hydrolyzing enzyme is selected from the group consisting of Dispersin B. PelAh, PslGh, Alginases, such as Alginase AI-II, Alginase AI-II′. Alginase AI-III, and any fusion proteins combining two or more proteins of said group. A preferred alginase is Alginase AI-II′.

In further embodiments, at least three, preferably at least four exopolysaccharde hydrolyzing enzymes as described herein are encoded by a nucleotide sequence in the genetically modified bacterium or oligonucleotide arrangement.

In preferred embodiments, the genetically modified Mycoplasma bacterium comprises as heterologous exopolysaccharide hydrolyzing enzymes Dispersin B, PelAh, PslGh and Alginate lyase AI-II′, and as a further heterologous protein the antimicrobial protein Lysostaphin as described in preceding embodiments.

It is evident that any combination of the herein envisaged gene products encoded by the one or more nucleotide sequences may be expressed as a fusion protein or a multitude of fusion proteins. In certain embodiments, the fusion protein comprises at least two gene products independently selected from the group comprising: exopolysaccharide hydrolyzing enzymes and antimicrobial proteins. In certain embodiments, the fusion protein further comprises an N-terminal secretion signal sequence as disclosed herein. In further embodiments, the secretion signal is the optimized MPN142 secretion signal sequence MKSKLKLKRYLLFLPLLPLGTLSLANTYLLQ (SEQ ID NO: 10).In alternative embodiments, the secretion signal is a mutagenized MPN142 secretion signal sequence having at least 80% sequence identity to SEQ ID NO: 10. preferably at least 85% sequence identity to SEQ ID NO: 10, more preferably at least 90% sequence identity to SEQ ID NO: 10, most preferably at least 95% sequence identity to

SEQ ID NO: 10. In certain embodiments, the fusion protein is LysAB2_SH3b. In certain embodiments, the fusion protein is Lysostaphin-Dispersin B.

In certain embodiments, the bacterium is a Mycoplasma pneumonia bacterium. Hence, in certain embodiments described throughout this specification, the Mycoplasma species subject of the invention have as genomic sequence a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, at least 97% global sequence identity to a naturally occurring Mycoplasma pneumoniae bacterium. In certain embodiments, the genetically modified Mycoplasma bacterium described herein has a genomic sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, at least 97% global sequence identity to reference M129-B7 Mycoplasma pneumoniae bacterium. In certain embodiments, the genetically modified Mycoplasma bacterium is M. pneumoniae M129 (-B7) (ATCC identifier 29342).

As illustrated in detail by the examples, the genetically modified Mycoplasma bacteria described herein and the pharmaceutical dosage units described herein are active against biofilms that are heterogenous in bacterial composition. Particularly, genetically modified Mycoplasma bacteria and pharmaceutical dosage units are active against a microbial biofilm formed (in part) by at least two distinct bacteria, such as Staphylococcus aureus and Pseudomonas aeruginosa.

In certain embodiments, the biofilm is formed in the respiratory system of said subject. In further embodiments, the biofilm is formed in the lower respiratory system (tract). In alternative further embodiments, the biofilm is formed in the upper respiratory system (tract). A skilled person understand the meaning of the term “respiratory system”, which may be annotated in the art as “respiratory apparatus”, or even “ventilatory system” in the art and is aware that the respiratory system comprises organs and structures used for gas exchange in animals, human being a non-limiting example hereof. In mammals such as humans, the upper respiratory tract includes the nose, nasal cavities, sinuses, pharynx and the part of the larynx above the vocal folds. In mammals such as humans, the lower respiratory tract includes the lower part of the larynx, the trachea, bronchi, bronchioles and the alveoli. In certain embodiments, said biofilm is formed in the lungs of said subject. In certain embodiments, said biofilm is formed in the trachea of the subject. In certain embodiment, the biofilm is formed in the bronchi and/or bronchiole of the subject. In preferred embodiments, the biofilm is a biofilm disposed on a tracheal tube which was earlier inserted in the respiratory tract of a subject (alternatively worded, the biofilm was developed on the tracheal tube after insertion into the subject). In further preferred embodiments, the biofilm is an endotracheal tube biofilm which is inserted in the respiratory tract of a subject. In further preferred embodiments, the biofilm is an endotracheal tube biofilm inserted in the trachea of a subject (i.e. and in vivo endotracheal tube). “Tracheal tube” is to be interpreted according to the generally accepted meaning in the art, i.e. a catheter that is inserted into the trachea for the primary purpose of establishing and maintaining a patient airway and to ensure the adequate exchange of oxygen and carbon dioxide. In certain embodiments, the tracheal tube is selected from the group consisting of: an endotracheal tube, a tracheostomy tube, or a tracheal button (tube).

In certain embodiments, the biofilm is a biofilm comprising hexosamine-containing polymers (PIA) is intended. Hexosamine-containing polymers (PIA) have been described in the art (for example in Kaplan et al., Genes involved in the synthesis and degradation of matrix polysaccharide in Actinobacillus actinomycetemcomitans and Actinobacillus pleuropneumoniae biofilms, Journal of Bacteriology, 2004). In certain embodiments, the biofilm comprises between 10% and 90% weight percentage, preferably between 20% and 80% weight percentage hexosamine-containing polymers. In certain embodiments, the biofilm comprises at least 10%, preferably at least 20%, at least 30%, at least 40%, at least 50%, at least 60% hexosamine-containing polymers. In certain embodiments, the biofilm comprises Pel and/or Psl. In further embodiments, the Pel and/or Psl present in the biofilm are P. aeruginosa Pel and/or Psl (Colvin et al., The Pel and Psl polysaccharides provide Pseudomonas aeruginosa structural redundancy within the biofilm matrix, Environ Microbiol, 2012). The protective role of alginate exopolysaccharide for Pseudomonas aeruginosa biofilms is known in the art (Leid et al., The exopolysaccharide alginate protects Pseudomonas aeruginosa biofilm bacteria from IFN-gamma-mediated macrophage killing. J Immunol, 2005). In certain embodiments, the use of an oligonucleotide arrangement as described herein for dispersing biofilms comprising Pel, Psl, and alginate exopolysaccharides is envisaged. In certain embodiments, the use of the genetically modified Mycoplasma bacterium or pharmaceutical dosage unit as described herein for dispersing biofilms produced by Pseudomonas aeruginosa (or a group of bacteria comprising or consisting essentially of P. aeruginosa) is intended. It is evident to a skilled person that biofilms commonly comprise a plethora of bacterial species. Hence, In certain embodiments, the use as described herein for dispersing biofilms produced by Staphylococcus aureus (or a group of bacteria comprising or consisting essentially of S. aureus) is envisaged. In certain embodiments, the use as described herein for killing and/or inactivating P. aeruginosa and S. aureus present in a microbial biofilm is intended. In certain embodiments, the use as described herein for reducing the growth rate of a biofilm comprising P. aeruginosa and S. aureus bacteria is intended. In further embodiments, the growth rate of the biofilm comprising P. aeruginosa and S. aureus bacteria is reduced by at least 10%, preferably at least 20%, at least 30%, at least 40%, at least 50%, at least 60%, at least 70%, at least 80%, at least 90%, or 100% when compared to a P. aeruginosa and S. aureus comprising biofilm not contacted with a nucleotide arrangement as described herein or a genetically modified bacterium comprising a nucleotide arrangement as described herein.

The genetically modified Mycoplasma bacteria described herein holds therapeutic value both in an inactivated or non-living state, and in a living form. When inactivated, the exopolysaccharide hydrolyzing enzymes described herein may be produced in a controlled environment. In certain embodiments, an antimicrobial protein is additionally produced by the bacteria. After inactivation of the producer bacteria, the inactivated (i.e. dead) bacteria may be administered to a subject (optionally as part of a pharmaceutical dosage unit) or the exopolysaccharide hydrolyzing enzymes may be enriched from a culture broth prior to administration to a subject. In their living state, the genetically modified Mycoplasma bacteria are introduced into a subject where they (continue to) produce the exopolysaccharide hydrolyzing enzymes (and optionally at least one antimicrobial protein) at the site of infection over a prolonged amount of time. In further embodiments, the live genetically modified Mycoplasma bacteria are able to propagate at or near the site of infection.

The term “attenuated” as described herein can be used interchangeably with terms such as “weakened” and “diminished”. The wording “attenuated strain” is commonly used in the art and refers to weakened disease agents, i.e. attenuated pathogens. An attenuated bacterium is a weakened, less vigorous, less virulent bacterium when compared to the traditionally occurring counterpart. An attenuated Mycoplasma bacterium according to embodiments of the invention is indicative for a genetically modified Mycoplasma bacterium wherein expression of genes whereof the gene product is responsible for a certain degree of virulence or toxicity have been modified in order to diminish or nullify the adverse effect of said gene on an infected subject. In certain embodiments, the genetically modified Mycoplasma bacterium described herein, which is preferably a Mycoplasma pneumonia bacterium, is 30% less toxic, preferably 40% less toxic, more preferably 50% less toxic, yet more preferably 60% less toxic, yet even more preferably 75% less toxic, most preferably 90% less toxic when compared to reference M129-B7 (ATCC identifier 29342) Mycoplasma pneumoniae bacterium.

A skilled person is well aware of methods to evaluate and quantify toxicity of bacterial strains. In the context of the present invention, a non-limiting examples of evaluating toxicity of the genetically modified Mycoplasma bacterium is measuring the inflammatory response in the lung, preferably by measuring inflammatory cytokines, measuring pulmonary lesions, and/or measuring haemorrhagic lesions in the mammary gland and/or lung of a test subject, preferably a mammal test subject such as but not limited to a rodent, non-human primate, or human.

While several Mycoplasma bacteria including M. pneumoniae are opportunistic pathogens, there are clear benefits of using an (artificially) attenuated Mycoplasma bacterium for therapeutic purposes such as dissolving bacterial biofilms. Firstly, the attenuating negates any potential uncertainty about any potential risk to develop an unwanted clinical image due to introducing the Mycoplasma bacteria into the patient. Furthermore, attenuating the Mycoplasma bacterium alleviates a medical practitioner and/or regulating authorities to assess the risk of using said bacterium in subjects having a further lowered immune system, either due to a severe manifestation of the bacterial infection, or due to the occurrence of comorbidities or underlying disease images.

The intended Mycoplasma genes of interest for attenuating the bacterium are indicated throughout this specification by their MPN (M. pneumoniae) number. A skilled person is aware that the MPN nomenclature is a standard manner of gene annotation in the technical field and that gene and/or protein names are readily derivable from publicly available resources such as the M. pneumoniae database http://mympn.crg.eu/essentiality.php or (academic) publications (including but not limited to Lluch-Senar et al., Defining a minimal cell: essentiality of small ORFs and ncRNAs in a genome-reduced bacterium, Molecular Systems Biology, 2015). It is evident that the MPN numbers are intended to also cover Mycoplasma genes from different strains, and it is thus evident for a skilled person that alternative annotations and classifications may be used to specify the same, or essentially the same genes. For example, a commonly used yet non-limiting system to annotate certain gene product is the IUBMB enzyme nomenclature. Reference works and tools to link certain enzymatic activities to specific IUBMB EC numbers are readily available in the art (e.g. McDonald et al., ExplorEnz: the primary source of the IUBMB enzyme list, Nucleic Acids Research, 2009). Hence, when reference herein is made to a certain MPN number, such references also encompass the corresponding enzymes in orthologue Mycoplasma bacteria categorized under the same IUBMB EC number. In certain embodiments, the attenuated Mycoplasma bacterium used to introduce the oligonucleotide arrangement has a (functional) modification such as but not limited to an inactivating mutation, deletion, and/or substitution in MPN133and/or MPN372.

In certain embodiments wherein the genetically modified Mycoplasma bacteria is comprised in a pharmaceutical dose unit further comprising at least one antimicrobial agent, said bacterium is comprised in a first composition and said antimicrobial agent is comprised in a second composition. In such embodiments, the pharmaceutical dosage unit may be considered to constitute a kit of parts comprising at least two components. In certain embodiments, at least the composition comprising the genetically modified Mycoplasma bacterium is a lyophilized composition that may need to be reconstituted prior to administration. In further embodiments, the pharmaceutical dosage unit or one or more of the compositions may be formulated into any suitable administration form, including but not limited to hard capsules, soft capsules, tablets, coated tablets such as lacquered tablets or sugar-coated tablets, granules, aqueous or oily solutions, syrups, emulsions, suspensions, ointments, pastes, lotions, gels, inhalants or suppositories, which may be provided in any suitable packaging means known in the art, non-limiting examples being troches, sachets, pouches, bottles, films, sprays, microcapsules, implants, rods or blister packs.

In alternative embodiments, the pharmaceutical dosage unit comprises a single composition comprising both the genetically modified Mycoplasma bacterium and the antimicrobial agent. In such embodiments, the antimicrobial agent may be present contained by and/or expressed by the bacterium or as an agent present in the composition as a further separate component.

In a further aspect, the invention is directed to a genetically modified (e.g. an attenuated) Mycoplasma bacterium as described herein or a pharmaceutical dosage unit described herein for use as a medicament. It is understood that a medicament as used in the context herein refers to a substance, or drug, that is used to diagnose, cure, treat, or prevent disease. Hence, in these aspects, the use of a genetically modified Mycoplasma bacterium is envisaged for the manufacturing of a medicament.

In certain embodiments, a genetically modified Mycoplasma bacterium as described herein or pharmaceutical dosage unit described herein for use in treating pneumonia is envisaged. Hence, in these aspects, the use of a genetically modified Mycoplasma bacterium is envisaged for the manufacturing of a pneumonia medicament. “Pneumonia” as used herein refers to an inflammatory condition of the lung affecting in particular the alveoli of the subject. The diagnosis of pneumonia is usually based on the assessment of physical signs, a chest radiograph, PCR-based methods, lung ultrasound, sputum cultures, or any combination thereof. Typical physical signs include but are not limited to low blood pressure, high heart rate, low oxygen saturation, increased respiratory rate, decreased chest expansion on the side affected by the pneumonia, bronchial breathing, crackling noises during inspiration, altered percussion of an affected lung, and increased vocal resonance. In certain embodiments, a genetically modified Mycoplasma bacterium as described herein or obtained by any of the methods described herein for use in treating Cystic Fibrosis (CF) is intended. Cystic fibrosis an autosomal recessive genetic disorder caused by a mutated CFTR gene that mainly affects the lung, while also affecting other organs such as the pancreas, liver, kidneys, and intestine. The main symptoms related to lung function are mucus build up, decreased mucociliary clearance and inflammation. These symptoms develop as a consequence of bacterial colonization and infection of the lungs of the patients. Non-limiting examples of bacteria responsible for lung infections in cystic fibrosis patients are P. aeruginosa, S. aureus, and Haemophilus influenzae. Often biofilms are formed in the lungs of cystic fibrosis patients due to presence of one or more of these bacterial species (Johnson et al., Novel understandings of host cell mechanisms involved in chronic lung infection: Pseudomonas aeruginosa in the cystic fibrotic lung, Journal of Infection and Public Health, 2019). In alternative further embodiments, a genetically modified (e.g. attenuated) Mycoplasma bacterium as described herein or obtained by any of the methods described herein for use in treating Chronic Obstructive Pulmonary Disease (COPD) is envisaged. Chronic obstructive pulmonary disease may be alternatively indicated by “chronic bronchitis” in the art and is an obstructive lung disease having a shortness of breath and cough with sputum production as main symptoms (Vogelmeier et al., Global Strategy for the Diagnosis, Management and Prevention of Chronic Obstructive Lung Disease 2017 Report: GOLD Executive Summary”. Respirology, 2017). In certain embodiments, the genetically modified (e.g. attenuated) Mycoplasma bacterium as described herein is used to treat subjects diagnosed with, or showing symptoms adequate to be diagnosed with pneumonia, preferably ventilator associated pneumonia, (recurrent) pneumonia as a consequence of cystic fibrosis, or pneumonia as a consequence of chronic obstructive pulmonary disease. In further embodiments, the subject having, or suspected to have pneumonia has a reduced standard lung volume of at least 10%, preferably at least 20%, preferably at least 30%, preferably at least 40%, preferably at least 50%, preferably at least 60%, preferably at least 70%, preferable between 70% and 90%, wherein the standard lung volume is selected from the group consisting of; tidal volume, inspiratory reserve volume, expiratory reserve volume, residual volume. In certain embodiments, the subject having, or suspected to have pneumonia has a reduced standard lung volume of between 10% and 95%, preferably between 10% and 50%, between 25% and 50%, between 50% and 95%, between 75% and 95%, wherein said standard lung volume is selected from the group consisting of; tidal volume, inspiratory reserve volume, expiratory reserve volume, residual volume. In certain embodiments, the subject having, or suspected to have pneumonia has a reduced standard lung capacity of at least 10%, preferably at least 20%, preferably at least 30%, preferably at least 40%, preferably at least 50%, preferably at least 60%. preferably at least 70%, preferable between 70% and 90%, wherein said standard lung capacity is selected from the group consisting of; inspiratory capacity, functional residual capacity, vital lung capacity, and total lung capacity. In certain embodiments, the subject having, or suspected to have pneumonia has a reduced standard lung capacity of between 10% and 95%, preferably between 10% and 50%, between 25% and 50%, between 50% and 95%, between 75% and 95%, wherein the standard lung capacity is selected from the group consisting of; inspiratory capacity, functional residual capacity, vital lung capacity, and total lung capacity. Standard lung volumes, standard lung capacities, and means to measure them have been described in detail in the art (Lufti, The physiological basis and clinical significance of lung volume measurements, Multidiscip Respir Med, 2017). A non-limiting method to assess the lung volumes and lung capacities described above is by spirometry. In further embodiments, a genetically modified (e.g. attenuated) Mycoplasma bacterium as described herein or obtained by any of the methods described herein for use in treating ventilator associated pneumonia is intended. It is understood that ventilator associated pneumonia is a type of lung infection occurring in patients subjected to mechanical ventilation breathing machines in hospitals (Michetti et al., Ventilator-associated pneumonia rates at major trauma centers compared with a national benchmark: a multi-institutional study of the AAST, J Trauma Acute Care Surg, 2012).

A further aspect of the invention concerns methods of treatment of a respiratory infection, such as bacterial respiratory infections involving P. aeruginosa and S. aureus, more preferably wherein said bacteria form a biofilm in the respiratory tract of a subject, wherein said method comprises a step of administering the genetically modified Mycoplasma bacterium or pharmaceutical dosage unit described in the present disclosure to said subject. In preferred embodiments, the administration step is an oral administration step. In certain embodiments, the genetically modified Mycoplasma bacterium and the one or more antimicrobial agents are administered simultaneously to the subject. In further embodiments, the genetically modified Mycoplasma bacterium and the one or more antimicrobial agents are administered simultaneously to the subject by different administration routes or methods. In alternative embodiments, the genetically modified Mycoplasma bacterium of the pharmaceutical dosage unit and the one or more antimicrobial agents of the pharmaceutical dosage unit are administered at distinct time points. In further embodiments, the administration of the antimicrobial agent is administered in a periodical or even continuous manner while the administration of the genetically modified Mycoplasma bacterium or pharmaceutical dosage unit comprising said bacterium occurs in a single administration step. In certain respiratory biofilm related embodiments, the administration step comprises inhalation of the genetically modified Mycoplasma bacterium or the pharmaceutical dosage unit.

In certain embodiments, a genetically modified Mycoplasma bacterium as described herein or obtained by any of the methods described herein for use in dissolving a (microbial) biofilm such as a biofilm produced by Pseudomonas aeruginosa and Staphylococcus aureus is intended. In certain embodiments, the microbial biofilm is dissolved by at least 10%, preferably at least 20%, preferably at least 30%, preferably at least 40%, preferably at least 50%, preferably at least 60%, preferably at least 70%, preferably at least 80%, preferably at least 90%, preferably at least 100% when compared to the dissolvement rate of a biofilm not treated by the genetically modified Mycoplasma bacterium such as the reference M129-B7 Mycoplasma pneumoniae bacterium. Methods to quantitatively assess biofilm growth are known to a skilled person (Haney et al., Critical assessment of methods to quantify biofilm growth and evaluate antibiofilm activity of host defence peptides, Biomolecules, 2018).

The terms “treat” or “treatment” encompass both the therapeutic treatment of an already developed disease or condition, such as the therapy of an already developed pulmonary disease, as well as prophylactic or preventive measures, wherein the aim is to prevent or lessen the chances of incidence of an undesired affliction, such as to prevent occurrence, development and progression of a pulmonary infection. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. “Treatment” can also mean prolonging survival as compared to expected survival if not receiving treatment.

As used herein, the terms “therapeutic treatment” or “therapy” and the like, refer to treatments wherein the object is to bring a subjects body or an element thereof from an undesired physiological change or disorder, including but not limited to pulmonary infections, to a desired state, such as a less severe or unpleasant state (e.g., amelioration or palliation), or back to its normal, healthy state (e.g., restoring the health, the physical integrity and the physical well-being of a subject), to keep it (i.e., not worsening) at said undesired physiological change or disorder (e.g., stabilization), or to prevent or slow down progression to a more severe or worse state compared to said undesired physiological change or disorder.

In certain embodiments, the pharmaceutical dosage unit further comprises one or more further pharmaceutical active ingredients in addition to the genetically modified Mycoplasma bacterium and optionally even in addition to the antimicrobial agent. In certain embodiments, the pharmaceutical dosage unit further comprises one or more non-active pharmaceutical ingredients or inactive ingredients, commonly referred to in the art as excipients.

The term “excipient”, commonly termed “carrier” in the art may be indicative for all solvents, including but by no means limited to: diluents, buffers (e.g., neutral buffered saline, phosphate buffered saline, or optionally Tris-HCl, acetate or phosphate buffers), solubilisers (e.g., Tween 80, Polysorbate 80), colloids, dispersion media, vehicles, fillers, chelating agents (e.g., EDTA or glutathione), amino acids (e.g., glycine), proteins, disintegrants, binders, lubricants, wetting agents, stabilizers, emulsifiers, sweeteners, colorants, flavorings, aromatisers, thickeners, agents for achieving a depot effect, coatings, antifungal agents, preservatives (e.g., benzyl alcohol), antioxidants (such as, e.g., ascorbic acid, sodium metabisulfite), tonicity controlling agents, absorption delaying agents, adjuvants, bulking agents (e.g., lactose, mannitol) and the like. The use of such media and agents for formulating pharmaceutical compositions is well known in the art. A skilled person is aware that when administration of a live Mycoplasma is envisaged to a subject in need thereof, the choice of excipients is limited to those that are considered not toxic or at least not toxic in the used concentrations for both subject and the bacterium.

In certain embodiments wherein a synthetic promoter is used, the expression of the one or more heterologous gene product is increased with at least 25%, preferably at least 35%, preferably at least 45%, preferably at least 50%, preferably at least 75%, preferably at least 100%, preferably at least 150%, preferably at least 200% compared to the expression level of the one or more heterologous gene product whereof expression is controlled by a naturally occurring promoter, preferably a naturally occurring M. pneumoniae promoter. In certain embodiments, the efficacy of a heterologous gene product as described herein part of a oligonucleotide arrangement as described herein is increased by at least 25%, preferably at least 35%, preferably at least 45%, preferably at least 50%, preferably at least 75%, preferably at least 100%, preferably at least 150%, preferably at least 200% when compared to said gene product under control of a naturally occurring promoter, preferably a naturally occurring M. pneumoniae promoter. Efficacy as used herein may be indicative of the potency of a heterologous gene product to dissolve and/or prevent formation of (microbial) biofilms, preferably wherein said biofilm is formed by P. aeruginosa and S. aureus.

An aspect of the invention is directed to a method of treating a subject diagnosed with, or suspected to have a pathogenic biofilm formation, wherein the method comprises a step of contacting the subject with a genetically modified bacterium as described herein or a pharmaceutical composition as described herein. In certain embodiments, the bacterium is a (live) Mycoplasma bacterium. In certain embodiments, the pathogenic biofilm formation is a respiratory biofilm formation. In further embodiments, the subject is diagnosed with, or suspected to have, ventilator associated pneumonia (VAP), Cystic Fibrosis (CF), or Chronic Obstructive Pulmonary Disease (COPD).

During further exploration and characterization of the genetically modified Mycoplasma bacteria described herein and accompanying pharmaceutical dosage units, the inventors unexpectedly observed that genetically modified bacteria comprising inactivating mutations in one or more genes which result in attenuating of toxicity of the bacteria (such as but not limited to MPN133 and/or MPN372) decrease the therapeutic potential of the final genetically modified Mycoplasma bacterium. Hence, without wishing to be bound be theory, while the use of non-pathogenic versions of certain bacteria are evidently of great interest for delivery and/or in vivo production of therapeutic proteins, the potential of said bacteria may be hampered by the lack of an adequate immune response towards the bacterium. For example, the present inventors have observed that the cytokine levels produced upon infection with an attenuated bacterium such as an attenuated Mycoplasma bacterium are markedly reduced, which leads to a minor activation of the immune system of the recipient and a reduction in clearance rate of the attenuated bacterium. In view hereof, the inventors have unexpectedly observed that supplementing the genetically modified bacterium with an antimicrobial agent may restore some of the therapeutic potential that is lost when inactivating (e.g. deleting) virulence factors in bacteria. Hence, inclusion of such an antimicrobial may be beneficial when harnessing certain bacteria as therapeutic vessels or therapeutic agents. A skilled person appreciates that “therapeutic agent” in the context of genetically modified bacteria such as those disclosed herein may refer to the bacterium as a whole, but also to certain therapeutic molecules (i.e. agents) comprised in, expressed by, and/or secreted by said bacteria.

In view of the above, a further aspect of the present invention concerns the use of an antimicrobial agent for increasing the in vivo potency of an attenuated bacterium as a therapeutic agent. Also envisaged is the use of an antimicrobial agent for increasing the in vivo potency of an attenuated bacterium as a delivery vessel for one or more therapeutic agents. The relative relationship between the antimicrobial agent and the bacterium is not particularly limiting, and therefore the antimicrobial agent can be a heterologous protein expressed by the bacterium but may equally be an antimicrobial agent which is added to the final pharmaceutical dosage unit. Yet alternatively, the antimicrobial agent may equally be brought in contact with the bacterium upon or even after administration of both components to a subject. In certain embodiments, antimicrobial agent is a bacteriocin, an antimicrobial peptide, an antibiotic, or any combination thereof. In further embodiments, the antimicrobial agent is selected from the group of bacteriocins as described herein, the group of antimicrobial peptides as described herein, the group of antibiotics as described herein, or any combination thereof. In certain embodiments, the attenuated bacterium is a bacterium that is modified, preferably genetically modified in such a manner that at least one virulence factor and/or at least one toxin is no longer functionally expressed.

“Functionally expressed” as used herein refers to the expression of a gene product, preferably a protein, in a state wherein said gene product is capable of exerting in full or at least in partial the function of the naturally occurring (i.e. unmodified, wild type) gene product. Hence, when in the context of the present invention reference is made to a gene product that is no longer functionally expressed, this may refer to the lack of any expression, or to expression of a modified (i.e. mutated) version of the gene product that is no longer capable of exerting its wild type function. When the gene product is a protein, this may refer to the expression of a truncated version of the protein or a mutagenized version of the protein.

In preferred embodiments, the antimicrobial agent is used to increase the in vivo potency of a Mycoplasma bacterium as a therapeutic agent. In alternative preferred embodiments, the antimicrobial agent is used to increase the in vivo potency of a genetically modified bacterium as a therapeutic agent. In further embodiments, the antimicrobial agent is used to increase the in vivo potency of a genetically modified Mycoplasma bacterium, preferably a Mycoplasma pneumoniae bacterium as a therapeutic agent. In yet further preferred embodiments, the genetically modified Mycoplasma pneumoniae bacterium is genetically modified to no longer functionally expressing the Ca2+ dependent cytotoxic nuclease gene (MPN133), and/or ADP-ribosyltransferase CARDS gene (MPN372). In a most preferred embodiment, the genetically modified Mycoplasma pneumoniae bacterium is genetically modified to no longer functionally express the Ca2+ dependent cytotoxic nuclease gene (MPN133), and/or ADP-ribosyltransferase CARDS gene (MPN372). Evidently, also envisaged are other Mycoplasma bacteria that are genetically modified to no longer functionally expressing orthologues of Ca2+ dependent cytotoxic nuclease gene (MPN133), and/or orthologues of ADP-ribosyltransferase CARDS gene (MPN372).

In certain embodiments, the use of the antimicrobial agent increases the in vivo (therapeutic) potency of the attenuated (e.g. genetically modified bacterium) by at least about 30%, preferably by at least about 40%, preferably by at least about 50%, preferably by at least about 60%, preferably by at least about 70%, more preferably by at least about 80%, most preferably by at least about 90% when compared to the corresponding genetically modified bacterium not containing, not in presence of, or not supplemented with the heterologous antimicrobial agent. In further preferred embodiments, the in vivo potency is restored to about the potency of the corresponding non-attenuated reference bacterium.

Preferably, the “in vivo potency” as referred to in the above embodiments indicates the in vivo potency of the genetically modified bacterium to act as a therapeutic agent against bacterial infections. Further preferably, the “in vivo potency” as referred to herein indicates the in vivo potency of the genetically modified bacterium to act as a therapeutic agent against bacterial infections of the respiratory tract. Yet further preferably, the “in vivo potency” as referred to herein indicates the in vivo potency of the genetically modified bacterium to act as a therapeutic agent against bacterial infections of the respiratory tract which are characterized by biofilm formation. Most preferably, the “in vivo potency” as referred to herein indicates the in vivo potency of the genetically modified bacterium to act as a therapeutic agent against bacterial infections of the respiratory tract characterized by biofilm formation, wherein the biofilm is formed by a group of bacteria comprising S. aureus and/or P. aeruginosa, preferably S. aureus and P. aeruginosa.

In a further preferred embodiment, the antimicrobial agent used to increase the in vivo potency of an attenuated bacterium as a therapeutic agent is an bacteriocin, preferably the bacteriocin lysostaphin. In certain embodiments, the bacteriocin is a bacteriocin having a sequence identity of least 65%, preferably at least 75%, more preferably at least 85%, most preferably at least 95% to SEQ ID NO: 1 and/or SEQ ID NO: 11.

In a highly specific embodiment, the antimicrobial agent is lysostaphin from Staphylococcus simulans and the genetically modified bacterium is a Mycoplasma pneumoniae bacterium wherein one or more virulence factors such as but not limited to MPN133 and MPN372 are no longer functionally expressed.

Thus, the present invention additionally envisages the use of lysostaphin for increasing the in vivo potency of an attenuated bacterium as a therapeutic agent effective against in vivo biofilms formed by S. aureus. The present invention therefore provides two solutions that render an attenuated Mycoplasma bacterium effective against in vivo biofilms formed by S. aureus: 1) incorporation of the genetic platform described herein that enables effectiveness against P. aeruginosa biofilms, or 2) incorporation of lysostaphin. Both embodiments “rescue” the in vivo potency of the attenuated Mycoplasma strain against S. aureus biofilms.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects, statements, and embodiments of the invention are further supported by the following non-limiting examples.

### EXAMPLES

In the present examples the inventors provide substantial experimental evidence for the findings disclosed herein and provide means to a skilled person to reproduce the findings of the current invention. By implementing state of the art techniques situated in the fields of systems biology and synthetic biology, a rationally engineered M. pneumoniae strains was generated that is able to target the biofilms formed by P. aeruginosa and S. aureus. Notably, this engineered Mycoplasma bacterium is particularly potent to overcome several drawbacks of current treatment strategies such as providing a solution to the large degree of heterogeneity of biofilms. Finally, the engineering Mycoplasma bacterium able to disperse both P. aeruginosa and S. aureus does not suffer from an increased metabolic load, which is routinely observed in the art when other bacteria are harnessed to act as an engineered therapeutic agent or deliver genetically encoded therapeutic agents.

Note that the genetically modified Mycoplasma bacteria described in the present Examples were generated using tailored engineering protocols developed by the inventors and described in detail in co-pending application PCT/EP2021/052110, of which the contents are inserted by reference into the present disclosure.

### 1. Efficacy of in Vitro and ex Vivo Dispersal Treatments of S. aureus Biofilm Formed on Plates and Catheters

Staphylococcus aureus is a frequent colonizer of medical implants and indwelling devices, such as catheters, to which the bacteria can be easily attached and produce a biofilm matrix. Biofilm architecture and composition differ greatly between in vivo and in vitro conditions (Bjarnsholt et al, 2013). To implement biofilm dispersal activity in Mycoplasma, we designed a genetic platform based on the mpn142Opt-derived secretion signal fused to a protein with antibiofilm activity (dispersin B). This platform was first transformed into CV2 cells (i.e. a Mycoplasma pneumoniae strain which does no longer express the Ca2+ dependent cytotoxic nuclease gene (MPN133) and ADP-ribosyltransferase CARDS gene (MPN372) as a result of extensive genetic engineering), generating the strain CV2-DispB. The CV2 strain is of particular interest due to the absence of any lesions caused by said strain upon introduction into a subject, as elaborated on in detail in earlier applications of the applicant.

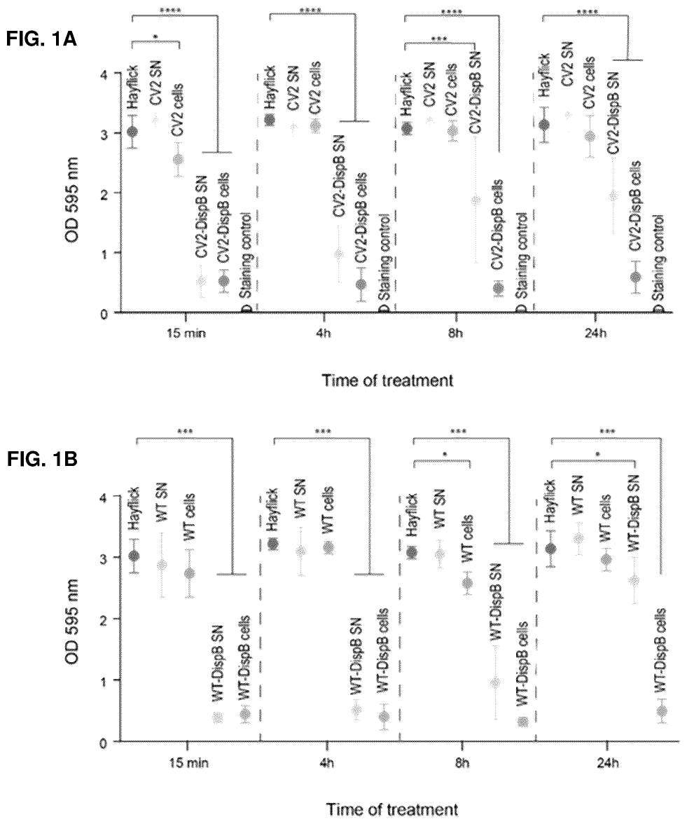

We tested the ability of CV2-DispB to dissolve S. aureus 24 h-mature biofilms in vitro by adding CV2-DispB supernatant (CV2-DispB SN) or directly adding CV2-DispB cells (CV2-DispB cells) to polystyrene wells containing the pre-formed biofilm (FIG. 1A). Staining with crystal violet showed that, at 15 min after treatment, both supernatant and cells-based treatments drastically reduced the biofilm integrity (FIG. 1A). Nonetheless, when the biofilm integrity was evaluated at longer exposure intervals, a mild decrease in effectiveness of CV2-DispB SN treatment could be observed, suggesting that the activity of secreted recombinant dispersin B is lost over time. In contrast, treatments based on CV2-DispB cells were highly effective throughout the experimental time, with a sustained reduction of the biofilm (FIG. 1A). Similar results were obtained when the platform was introduced in the WT strain (FIG. 1B).

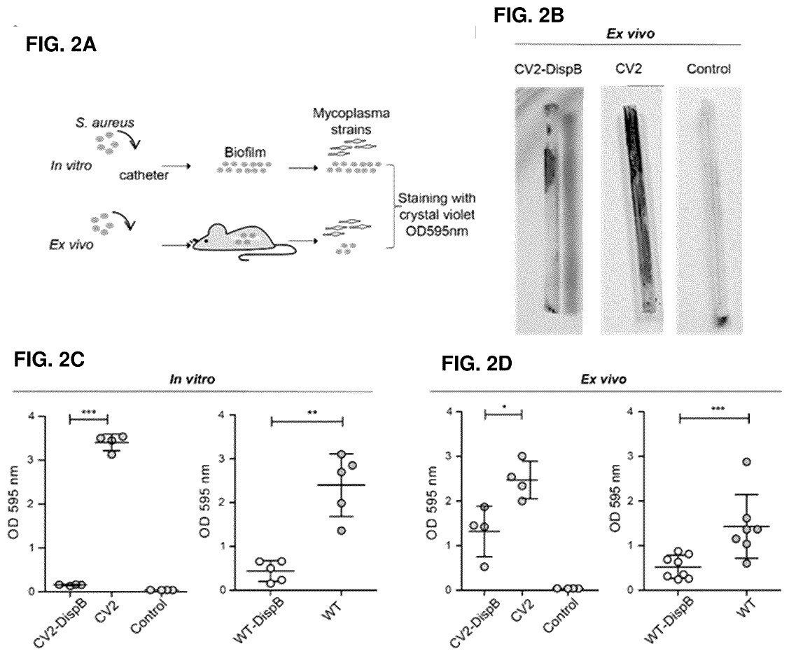

We further tested the usefulness of our M. pneumoniae dispersin B platform against either in vitro or in vivo formed S. aureus biofilm developed on catheters (FIG. 2A). The S. aureus infected catheters were surgically excised from mice and treated ex vivo for 4 h with CV2-DispB cells, using the CV2 strain as a control, and biofilm dispersion was quantified by crystal violet staining. A representative picture of the catheters after the ex vivo treatment is shown (FIG. 2B). The catheters treated with CV2-DispB strains, but not those treated with CV2, showed a significant dispersion of biofilms formed not only in vitro (FIG. 2C) but also formed in vivo and subsequently treated ex vivo (FIG. 2D). Similar results were obtained when comparing the WT-DispB to the WT strain (FIGS. 2C and 2D). Overall, these results highlight the capacity of the CV2-DispB attenuated chassis to disrupt S. aureus biofilms, not only in vitro but also ex vivo.

### 2. In Vivo Treatment of Mice Carrying Catheters Colonized by S. aureus

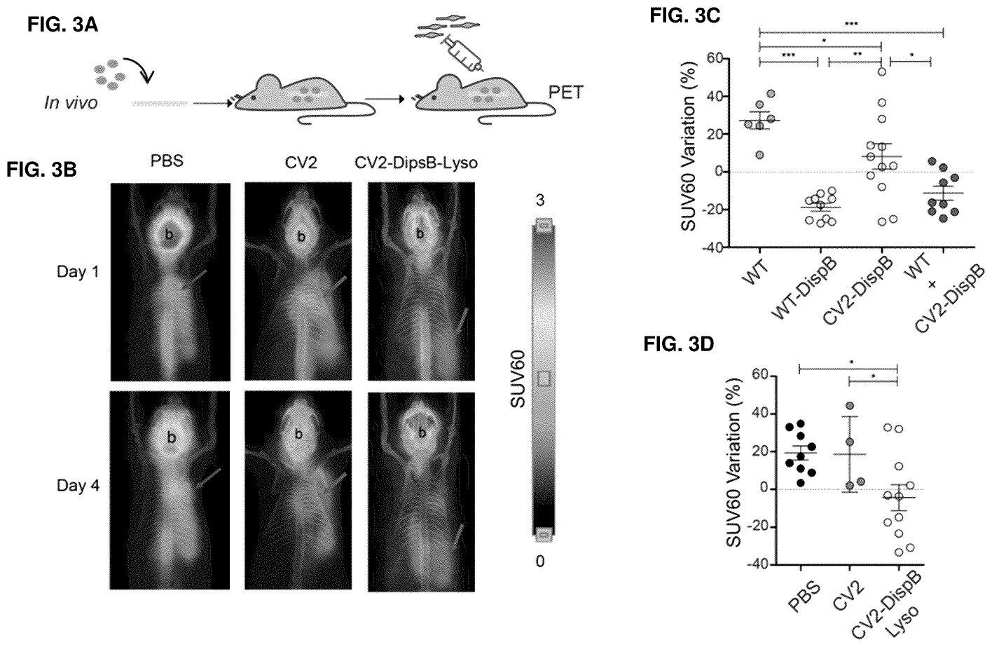

Next, we examined whether WT-DispB and/or CV2-DispB cells were able to dissolve S. aureus biofilms in vivo. To this end, CD1 mice carrying subcutaneous catheters with biofilms developed in vivo were treated subcutaneously with a single dose of different M. pneumoniae strains administered subcutaneously into the surrounding area of the catheter (FIG. 3A). Mice treated with CV2, WT or PBS were used as negative controls. Each group was assayed twice in separate experiments, to ensure the reproducibility of the model and obtain statistically robust results. All animals were analyzed by positron tomography with [¹⁸F]-FDG-MicroPET, at both 1- and 4-days post-treatment, as detailed previously (Garrido et al, 2014) (FIG. 3). To obtain a qualitative estimate of the bacterial abundance, immune cell infiltration and inflammation, we determined the SUV60 signal (see Methods). The efficacy of the treatment was expressed as the percentage of variation of the SUV60 signal obtained in the catheter arca from 1 to 4 days after treatment. In line with previous results (Garrido et al, 2014), mice treated with PBS showed an increased SUV60 signal of around 20%, indicative of the virulent progress of the S. aureus biofilm infection. Administration of WT or CV2 did not induce any therapeutic effect, showing 25% and 20% of SUV60 increasing values, respectively (FIGS. 3C and 3D). In contrast, all mice treated with WT-DispB cells showed a significant (p<0.001) decrease of around 20% in the PET signal (FIG. 3C), a drop that according to previous studies can be associated to a decrease of around two orders of magnitude of S. aureus living cells (Garrido et al, 2014). Unexpectedly, the therapeutic effect of WT-DispB strain was only partially reproducible (i.e., to a lesser consistent degree, albeit still statistically significant) in mice treated with the CV2-DispB strain (FIG. 3C). Since the in vitro and ex vivo treatments (discussed above) showed that CV2-DispB was as effective as WT-DispB to disperse biofilms in vitro, we investigated whether the lesions and/or the inflammatory response evidenced by WT in the mammary gland model played a complementary role in the efficacy of our M. pneumoniae platform. For this, we included one group of CD1 mice treated with a mix (1:1) of WT and CV2-DispB strains (FIG. 3C). While these two strains individually were unable (WT) or only partially able (CV2-DispB) to efficiently disperse S. aureus biofilms, their co-administration resulted in a treatment as efficient as that with the strain WT-DispB (FIG. 3C), indicating that the inflammatory response triggered by Mpn133 and Mpn372 proteins might have a role in clearing the infection. Altogether, these results suggested that in situ release of dispersin B by the CV2 strain can be sufficient, but fully reliable to control an S. aureus catheter infection in vivo.

### 3. Identification of an Antimicrobial Agent such as Lysostaphin to Obtain Robust S. aureus Biofilm Dispersion by the CV_DispB Strain in Vivo

To avoid any requirement for an inflammatory response in order to remove an S. aureus infection, we tested if expressing a bacteriolytic agent in combination with dispersin B in the CV2 strain could remove S. aureus infection, without the adverse (i.e. pathogenic) effects that are encountered when using a WT strain. As a non-limiting example, the glycylglycine endopeptidase lysostaphin was selected, which cleaves the pentaglycine crossbridge of the staphylococcal cell wall, killing S. aureus and affecting biofilms in vitro and in vivo (Wu et al, 2003; Kokai-Kun et al, 2009).

First, we characterized the expression of lysostaphin by M. pneumoniae. For this, we obtained different strains by introducing the lysostaphin coding gene under naturally-existing (Pmpn665 and Pmg438) promoters, or a set of different synthetic (P1 to P5) promoters, whose design was based on the rules governing efficient transcription and translation in this bacterium (Yus et al., 2017). We then checked the bacteriolytic activity of different strains on S. aureus growth curves. Strains with endogenous promoters showed minor effects on S. aureus growth. In contrast, we identified the P3 synthetic promoter as the one producing the highest lysostaphin activity, as inferred from the drastic effect of the strain carrying this promoter in S. aureus growth.

We next transformed CV2-DispB cells with a transposon vector harbouring the P3_mpn142Opt_lysostaphin construct, to generate the CV2-DispB-Lys strain. The ability of this strain to disrupt the progression of S. aureus biofilms in vivo was assessed as before by [¹⁸F]-FDG-MicroPET images in CD1 mice carrying colonized catheters. Indeed, mice treated with CV2-DispB-Lys cells showed impaired biofilm progression with respect to those receiving CV2-DispB or CV2 (FIGS. 3C and 3D). These results confirmed that the attenuated CV2 strain could represent an attractive chassis vector against S. aureus biofilms if the expression of a biofilm dispersal agent is combined with an antimicrobial peptide. Without wishing to be bound by theory, it is hypothesized that alternative antimicrobial agents may also be suitable for this purpose.

### 4. Evaluation of Metabolic Load on a Mycoplasma Strain upon Introducing Multiple Heterologous Proteins for Expression

It has been described in the art at numerous instances that genetically engineering bacteria to produce foreign (i.e. heterologous) proteins or gene products may be detrimental for the proper functioning of the metabolism of the organism (e.g. Marschall et al., Tunable recombinant protein expression in E. coli: enabler for continuous processing?, Appl Microbiol Biotechnol. 2016).

As described in the art, the main challenges of recombinant protein production in e.g. E. coli are associated with non-suitable (too high or too low) levels of recombinant expression:
1. First, high-level expression and the presence of foreign plasmids drain the hosts' metabolic resources (often referred to as metabolic load or metabolic burden) (Bentley et al. 2009; Bienick et al. 2014; Glick 1995; Mairhofer et al. 2013). Metabolic load often resulting in depletion of amino acids or aminoacyl-tRNAs and triggering heat-shock response can therefore ultimately affect product quality-related issues (specific activity, stability, and immunogenicity) and product quantity-related issues (product degradation, lower specific product yields, lower biomass yields, or shorter culture stability).
2. Second, high-level expression of recombinant products can lead to the formation of unfolded or partially folded insoluble protein aggregates known as inclusion bodies which show no catalytic function or activity (Baig et al. 2014; Kane and Hartley 1991).
3. Third, the production of many recombinant products, especially proteins containing disulfide bridges, demands translocation between compartments of the E. coli cell factory (Baneyx and Mujacic 2004). Here, too high levels of recombinant protein expression can lead to the blocking of translocation pathways.

The mentioned challenges are either fully or to a great extent caused by recombinant protein expression. The level of recombinant protein expression is affected by the strength of the expression system which involves the strength of the promoter used and the plasmid copy number (Keasling 1999) as well as the process technological parameters such as temperature and the specific growth rate (Hellmuth et al. 1994; Rodríguez-Carmona et al. 2012).

Finally, it is frequently observed that a reduction of the protein expression level leads to increased end product titers, since the cells can be maintained in a productive state for a longer time (Sagmeister et al. 2014; Sagmeister et al. 2013b).

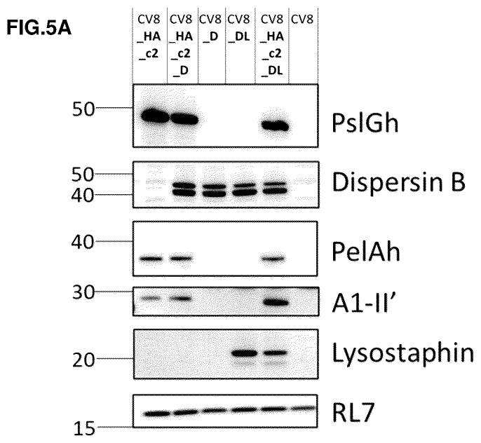

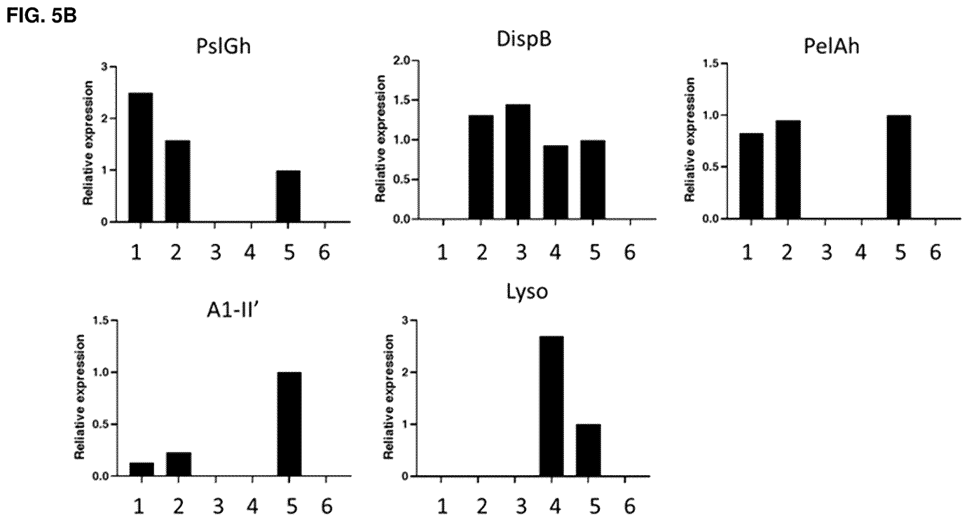

In view of these concerns, we examined whether the degree and extent of genetic modification had a negative impact on the expression levels of the recombinant proteins. The main results are depicted in FIG. 5 and summarised below for each recombinant protein:
PslGh: The strain with only one inserted genetic platform shows higher levels of PslGh than strains having the two genetic platforms. Interestingly, when the platform is including two genes (DL; sample 5) the levels are lower than when it includes only one additional gene (D; sample 2). However, this observation could not be seen in the same two strains for e.g. Dispersin B or PelAh expression levels.
Dispersin B: Each clone shows a similar expression level. Arguably sample 3 (corresponding to strain CV8_D) showed the highest Dispersin B expression level, followed by sample 2 (that has the additional genetic platform HA). Samples 4 and 5 were characterised by a lower expression which may be associated with the lysostaphin protein. However, this difference is not significant.
PelAh: PelAh expression levels appear consistent throughout the different samples (i.e. strains). There is no correlation with the number of genetic constructs and hence the recombinant proteins that are expressed.
AI-II′: The strain with two genetic platforms (sample 5) and thus effectively expressing 5 different proteins is the one that shows the highest levels of expression. Sample 2, corresponding to a strain with also two genetic platforms but only expressing 4 proteins shows significantly lower expression levels of AI-II′. Thus, the difference can by no means be attributed to the number of genetic platforms included in the strain.
Lysostaphin: The strain with one genetic platform shows the highest levels of expression.

For PslGh and lysostaphin, the highest level of expression seems to be related with having only one platform inserted. But for AI-II′ the opposite reasoning applies. Furthermore, expressing one gene or several does not seem to have an impact in Mycoplasma when looking at Dispersin B expression levels which showed similar levels of expression regardless of the further genetic background of the strain. Hence we can conclude that surprisingly and in contrast to what is generally accepted in the art.

Mycoplasma does not appear to suffer from a notable degree of metabolic burden due to introducing genetic constructs and expression of their gene product. Hence, engineered Mycoplasma bacteria might be particularly suited to act as therapeutic agents, or as delivery vessel for producing and transporting therapeutic agents.

### 5. Genetically Modified Mycoplasma Bacteria able to Disperse Multiple Bacteria in a Biofilm are more Potent than Combining Separate Genetically Modified Mycoplasma Bacteria that each Target a Single Biofilm Bacterium

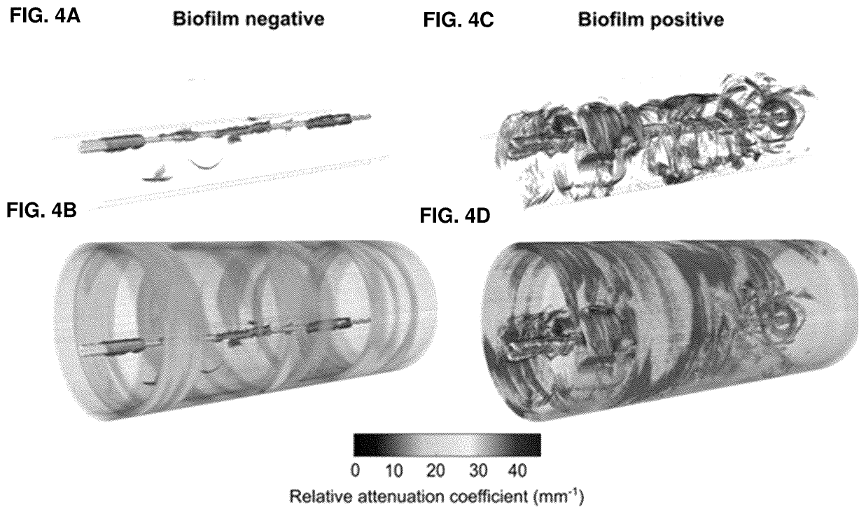

The heterogeneity in composition between different spatial localisations of a single biofilm-covered area is a further difficulty for treatment strategies (e.g. described in Dsouza et al., In vivo detection of endotracheal tube biofilms in intubated critical care patients using catheter-based optical coherence tomography, Journal of Biophotonics, 2019, also depicted in FIG. 4), as this heterogeneity may allow biofilm areas having a high concentration of particular bacteria to survive treatment strategies that are directed to a single aspect, i.e. bacterium, of the biofilm.

The genetically modified Mycoplasma bacteria described herein are particularly useful for solving this problem, since the therapeutic agents will be evenly distributed across different biofilm areas. There is no need for the (near simultaneous) presence of multiple therapeutic agents or genetically modified bacteria targeting a single bacterium to disperse any given location of the biofilm.

### 6. Activity of the Genetically Engineered Mycoplasma Bacteria on Mixed Biofilms

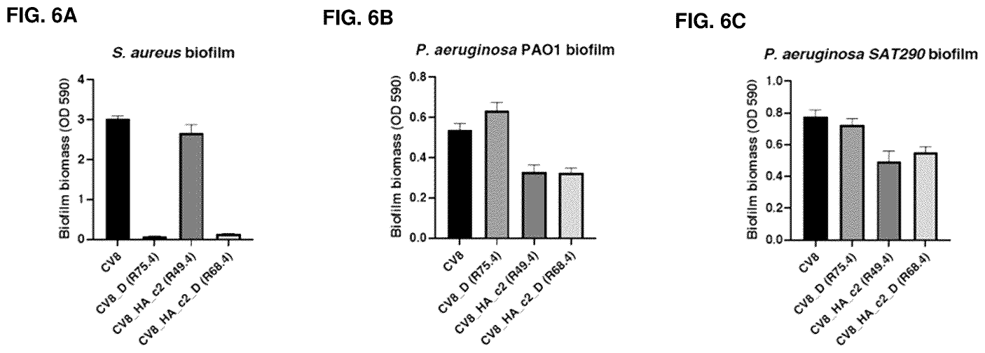

The dispersal activity of engineered strains was assessed in a crystal violet assay, of which the results are depicted in FIG. 6.

We could observe that S. aureus biofilm (FIG. 6A) is degraded by R75.4 and R68.4 strains; both expressing the Dispersin B protein. The CV8 and R49.4 strains are unable to degrade the biofilms because they are not expressing Dispersin B. Additionally, we could observe that biofilms formed by PAO1 and SAT290 P. aeruginosa strains (FIGS. 6B and 6C) are degraded by strains having the HA platform. Therefore we conclude that the strain expressing dispersal proteins against S. aureus and P. aeruginosa is active in all the tested biofilms.

To further assess the potency of the final engineered Mycoplasma strain(s) in a more complex environment mimicking a clinically relevant situation, we set up the conditions to form a mixed biofilm, which is composed by both S. aureus and P. aeruginosa mixed in different ratios (1:10; 1:1; 10:1). We observed that, independently of the ratio, the presence of P. aeruginosa has an advantage on the biofilm, indicated macroscopically by the colour of the biofilms and by the optical density of the untreated biofilm (data not shown), so we choose a ratio 10:1 for the next assay.

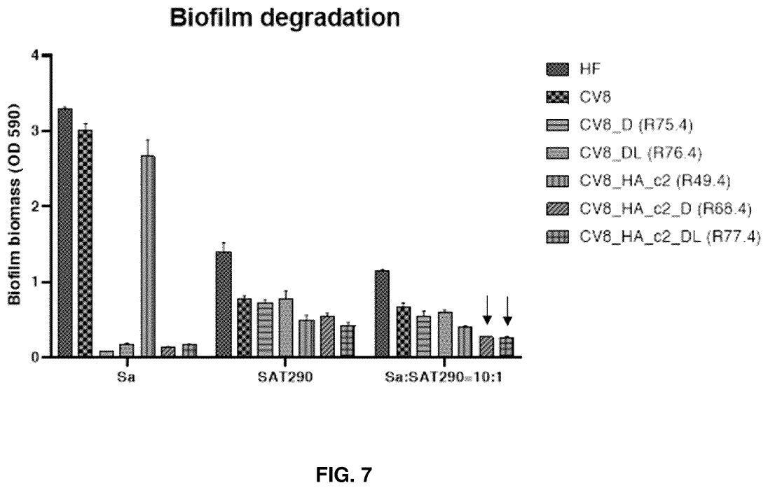

Next, we analysed the capability of the M. pneumoniae strains to degrade the biofilms in a crystal violet assay (FIG. 7).

The mixed biofilm formed by S. aureus and P. aeruginosa strains SAT290 in a ratio 10:1 shows the same degradation profile when treated with the strain CV8_D, CV8_DL or CV8_HA, indicating that this ratio allows the formation of a biofilm where P. aeruginosa and S. aureus are in similar proportions.

Interestingly, this mixed biofilm is best degraded when treated with the supernatant of the strains CV8_HA_c2_D (R68.4) and CV8_HA_c2_DL (R77.4) (conditions indicated with arrows in FIG. 7).

Thus, these results demonstrate that the treatment with a M. pneumoniae strain that expresses platforms against both S. aureus and P. aeruginosa is more effective than the treatment with a strain targeting a single pathogen.

7. Combination of the Genetically Modified Mycoplasma Strains with Antibiotics

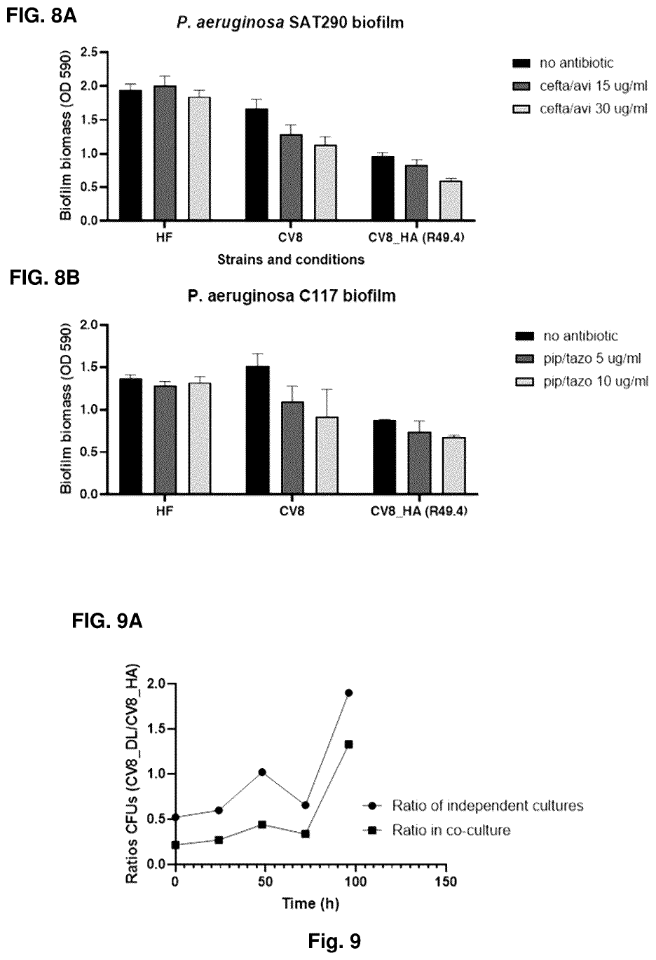

We studied a possible increase of the biofilm dispersal activity of CV8_HA strain, that expresses dispersal agents against P. aeruginosa, when combined with antibiotics. We used two different clinical isolates of P. aeruginosa (SAT290 and C117) and two different standard of care antibiotics (Ceftazidime-Avibactam and Piperacillin-Tazobactam). First, we studied the effect of different concentrations of these antibiotics on the growth of P. aeruginosa isolates and M. pneumoniae. While M. pneumoniae is naturally resistant to these antibiotics, we identified the minimal concentrations (>15 μg/ml for cefta/avi and >5 μg/ml for pip/tazo, respectively) that kills P. aeruginosa cells when grown in suspension, but that has no effect when cells are organized in a biofilm. Therefore, we studied the degradation of the biofilm of the P. aeruginosa clinical isolates when exposed to the supernatant of CV8_HA strains with or without these antibiotics (FIG. 8). We observed that the supernatant of CV8_HA strain combined with the antibiotics has an increased dispersal activity. Interestingly, we also found an unexpected result: the empty CV8 strain, not expressing the dispersal agents, can also partially dissolve the biofilm in combination with antibiotics, even though less efficiently than CV8_HA, suggesting that the supernatant from any M. pneumoniae strain contains some secreted components that acts on P. aeruginosa biofilm.

These data indicate that the activity of standard of care antibiotics that are not effective on a P. aeruginosa biofilm can be rescued by combining them with the heterologous proteins secreted by CV8_HA strain.

### 8. Two Independent Genetically Engineered Strains may Compete and Change the Relative Amounts of Therapeutic Payload Released in a Subject

We hypothesised that when co-administering 2 or more genetically engineered Mycoplasma strains (such as but not limited to Mycoplasma pneumoniae strains), there is a risk that one strain outcompetes the other strain(s), leading to an unwanted change in amount of therapeutic proteins that are produced in a subject in need thereof. This hypothesis was evidenced by an experiment wherein the propagating of two individual engineered strains having a combined set of therapeutic proteins that are required to be active against biofilms comprising P. aeruginosa and S. aureus was assessed over a prolonged time span. In brief, the two engineered strains were plated either on separate growth plates or in a single growth plate and the CFU ratio was determined for each condition.

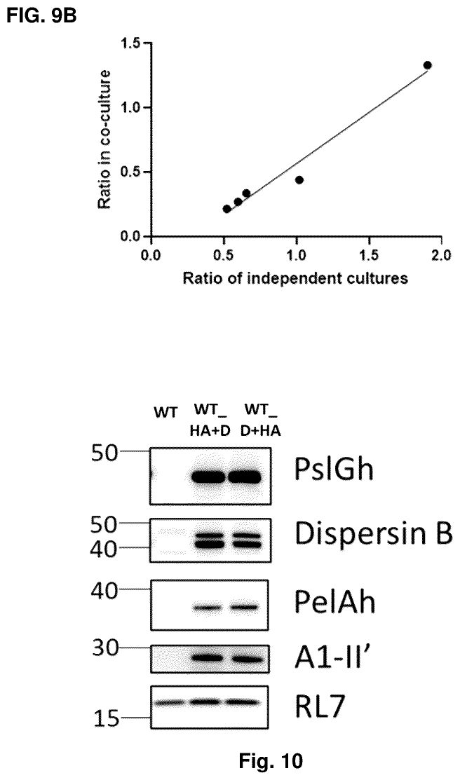

As depicted in FIG. 9(A), a drastic disturbance of the initial ratio could be observed after approximately 3 days of culture, wherein the growth of one strain starts to dominate the growth of the other strain (regardless of whether these were cultured in conditions of direct competition for nutrients or not). Thus, when co-administering a combination of genetically engineered strains in a subject, prolonged presence of said strains will become skewed and the relative amounts of therapeutic proteins will be disturbed, leading to at least a suboptimal treatment outcome. Furthermore, the experiments showed a convincing correlation between the two growth strategies FIG. 9(B) (R²=0.98). Thus, the experiment shows that when using multiple strains, certain strains may become dominant when cultured in a single environment for a prolonged period of time.

In conclusion, this experiment unambiguously demonstrates yet a further beneficial effect of the engineered Mycoplasma strains described herein that are able to deliver a combination of therapeutic proteins without needing to rely on co-administration of different strains. Hence, the engineered Mycoplasma strains described herein will have improved potency vis-à-vis any combination treatment strategy described in the art, the latter being prone to showing changes in the relative amounts of therapeutic proteins that are delivered into a subject over time.

### 9. The Order of Transformations does not Impact in the Activity of the Product Strain

No significant differences in terms of expression levels of the 4 heterologous proteins could be observed between strains that have been genetically modified in different sequential order (FIG. 10). Hence, one can conclude that the sequential order of the transformation of the different genetic platforms does not affect to the levels of expression of heterologous proteins, and any effects that are observed are not caused by clonal variability.

### 10. Rescue of the in Vivo Anti S. aureus Biofilm Activity of the Engineered M. pneumoniae Strain

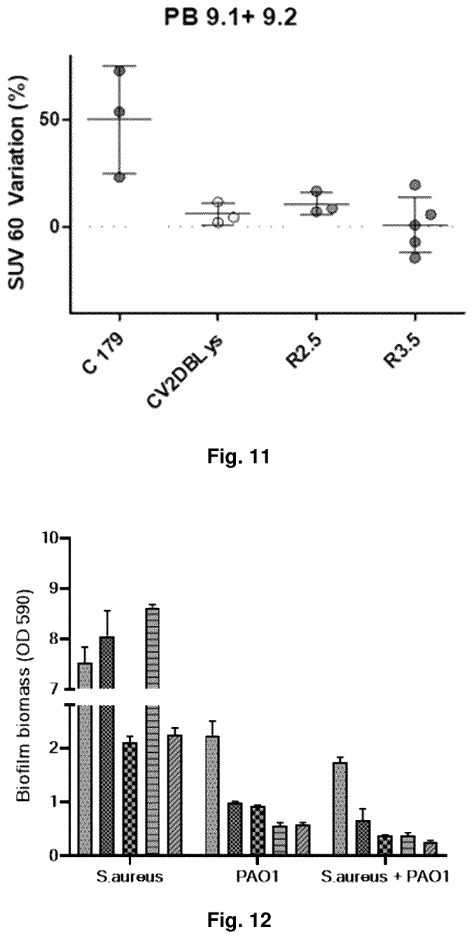

The anti-biofilm activity of the strains CV2_DispLyso; PB_Ch3_AI-II′_PelAh_Pslh_DispB and PB_Ch3_AI-II′_PelAh_Pslh_DispB_Lyso in S. aureus biofilms formed in vivo in catheters. Surprisingly, no differences between CV2_DispLyso and PB_Ch3_AI-II′_PelAh_Pslh_DispB could be observed, suggesting that in vivo the addition of the platform for counteracting P. aeruginosa can compensate the lack of lysostaphin for the degradation of the biofilm. Also, similar levels of degradation of biofilm with the strains PB_Ch3_AI-II′_PelAh_Pslh_DispB_Lyso (R3.5) and PB_Ch3_AI-II′_PelAh_Pslh_DispB (R2.5) could be observed (FIG. 11), suggesting that the addition of lysostaphin in the final engineered Mycoplasma strain is not critical and S. aureus biofilm dispersion can be obtained in vivo with an engineered strain that expresses these 4 enzymes involved in degradation of biofilm. Thus, the lack of lysostaphin in the final product does not affect the biofilm degradation. It means that the additional platform added for degrading biofilms of P. aeruginosa unexpectedly compensates the absence of lysostaphin gene.

### 11. Additional Experiment to Asses in Vitro Efficacy in Complex Biofilms

A further experiment was conducted to compare different strains in biofilms formed by S. aureus, P. aeruginosa (PAO1) or complex biofilms (S. aureus+PAO1). FIG. 12 depicts the results of said experiment. As expected, the strain expressing both platforms (PB_Ch3_HA_Disp, the outer right sample for each condition) is more effective than a strain expressing DispB (CV8_Disp; designed to degrade biofilms formed by S. aureus) or strain engineered to destroy biofilms formed by P. aeruginosa PAO1 strain (PB_Ch3_HA).

### 12. Additional experiment to demonstrate in vivo efficacy of the final engineered strain

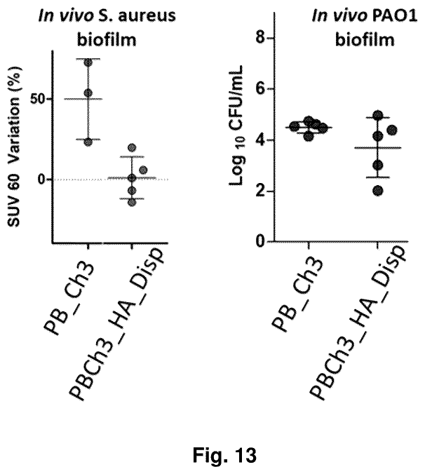

It could be observed that (FIG. 13, left panel) the final product strain (PB_Ch3_HA_Disp) was able to destroy biofilms formed by S. aureus in a catheter in vivo. The strain maintained effective against a biofilm formed by P. aeruginosa in a catheter in vivo (FIG. 13, right panel).

### 13. Summary of Used Strains Throughout the Experiments

The strains used in the experiments described herein are the following:
CV2: Chassis Mycoplasma pneumoniae strain which does no longer express the Ca2+ dependent cytotoxic nuclease gene (MPN133) and ADP-ribosyltransferase CARDS gene (MPN372) as a result of extensive genetic engineering.
CV2_DispB: chassis strain CV2 transformed with the minitransposon vector to express and secrete the Dispersin B gene.
CV8: Chassis strain derived from CV2 where the mpn051 was substituted by gspA gene.
CV8_D (R75.4)*: chassis strain CV8 transformed with the minitransposon vector to express and secrete the Dispersin B gene.
CV8_DL (R76.4)*: chassis strain CV8 transformed with the minitransposon vector to express and secrete the Dispersin B and Lysostaphin proteins.
CV8_HA_c2 (R49.4)*: Chassis CV8 transformed with a transposon vector that allows the expression of AI-II′ alginate lyase and two hydrolases PelAh and PslGh. Isolated clone with the resistance marker removed.
CV8_HA_c2_D (R68.4)*: CV8 strain expressing Dispersin B, AI-II′, PelAh and PslGh. It is the 49.4 strain transformed with the dispersin platform. It is a pool of cells. Not single clone isolated.
CV8_HA_c2_DL (R77.4)*: CV8 strain expressing Dispersin B, Lysostaphin, AI-II′, PelAh and PslGh. It is the 49.4 strain transformed with the Dispersin B and Lysostaphin platform. It is a pool of cells. Not single clone isolated.
WT_D (R69)*: wild type strain expressing Dispersin B. Pool of cells.
WT_HA_D (R64.4)*: wild type strain first transformed with platform HA and then Dispersin B. Pool of cells.
WT_D_HA (R65.4)*: wild type strain first transformed with platform Dispersin B and then with HA platform. Pool of cells.
PB_Ch3_AI-II′_PelAh_Pslh_DispB_Lyso: strain expressing Dispersin B, AI-II′, PelAh, PslGh, and Lysostaphin (alternative strain of R77.4).
PB_Ch3_AI-II′_PelAh_Pslh_DispB: strain expressing Dispersin B, AI-II′, PelAh, and PslGh (alternative strain of R68.4).
PB_Ch3: CV8 derived strain with additional genes depleted (mpn686 and mpn257). Also, the gp35 recombinase and resistance markers have been depleted.
PB_Ch3_HA_Disp: PB_Ch3 platform further transformed with Dispersin B and HA platform.
*=internal reference numbers.

Engineered Mycoplasma PB_Ch3 strain is deposited under the Budapest Treaty at the Spanish Type Culture Collection “Colección Española de Cultivos Tipo” (CECT) (Valencia, Spain) under No. xxxx on Sep. 20, 2022.

Engineered Mycoplasma PB_Ch3_HA_Disp strain is deposited under the Budapest Treaty at the Spanish Type Culture Collection “Colección Española de Cultivos Tipo” (CECT) (Valencia, Spain) under No. yyyy on Sep. 20, 2022.

### 14. Material and methods

In Vitro Dispersal Assay of Catheter-Associated S. aureus Biofilms

Sealed implants and S. aureus mature biofilms were prepared as previously reported (Garrido et al., 2014). Briefly, commercial Vialon® 18G 1.3-by 30-mm catheters were cut into 20-mm segments and sealed under sterile conditions with petrolatum and tissue glue. Cleaning and disinfection were achieved thereafter by immersion in DD445 and ethanol (15 min in each solvent). Sterility of catheters was checked by absence of turbidity after 24-h incubation (37° C.) in TSB. To establish mature biofilms, sterile catheters were immersed in 6-well polystyrene plates containing 1 ml of a suspension containing ˜1×106 CFU of S. aureus in TSB-glc and incubated at 37° C. for 24 h. After that, catheters were rinsed with 1 ml of Hayflick medium and treated for 4h, at 37° C., with 1 ml of ˜1×108 CFU of mycoplasma cells. The biofilms attached to the catheters were stained with crystal violet 0.1% (15 min, room temperature) and subsequently destained with ethanol: acetone (80:20, vol/vol). The resulting solution was quantified by OD 595 nm in a Multiscan microplate reader. As a staining control, catheters non-infected with S. aureus were treated with 1 ml of Hayflick and processed in a similar manner.

### Ex Vivo Dispersal Assay of Catheter-Associated Biofilms

Catheters carrying S. aureus biofilms were generated as described above, and immediately implanted subcutaneously in anaesthetised mice through a minimal surgical incision in the interscapular area. After 18 h, animals were euthanized to remove aseptically the catheters, which were individually rinsed with PBS, treated (37° C., 4h) with 1 ml of 1×108 CFU of mycoplasma cells and processed as described above. Also, a staining control based on catheters non-infected with S. aureus was included.

### In Vivo Evaluation of Catheter-Associated Biofilms Treatment by [¹⁸F]-FDG-Micro-PET

To monitor infection by [¹⁸F]-FDG-MicroPET imaging, as previously detailed (Garrido et al, 2014), fasted mice were anesthetized with 2% isoflurane in O2 gas inhalation and intravenously injected with 18.8-1.9 MBq of [¹⁸F]-FDG. After 1h of radiotracer uptake under continuous anaesthesia, PET images were taken in a small-animal tomography apparatus (MicroPET) by laying mice in a prone position and capturing images for 15 min. Images were reconstructed using a true three-dimensional (3D) Ramla algorithm reconstruction with 2 iterations and a relaxation parameter of 0.024 into a 128 by 128 matrix with a 1-mm voxel size, applying dead time, decay, random, and scattering corrections. For [¹⁸F]-FDG uptake assessment. MicroPET images were analysed using the PMOD software, and semiquantitative results were expressed as the standardized uptake value (SUV) index, obtained by normalization with the formula SUV=[(RTA/cm3)/RID]×BW, where RTA is the radiotracer tissue activity (in becquerels), RID is the radiotracer injected dose (in Bq), and BW is the mouse body weight (in grams). After qualitative inspection of the images, volumes of interest (VOI) were manually drawn on coronal 1-mm-thick consecutive slices including the entire catheter area. For catheter image quantification, to avoid manual bias of surrounding areas, a new VOI was generated semi-automatically using the threshold of 60% of maximum pixel for SUV mean calculation (SUV60 index). The results were expressed as the % of SUV 60 increase calculated as follows: [(SUV60 at day 4×100)/SUV60 at day 1]−100.

Dispersal Assays of Biofilms Performed in Vitro in multiwell plates

an overnight s. Aureus culture was diluted 1:40 in tsb-glc, and dispensed (100 μL/well) in 96-well polystyrene microtiter plates. Plates were incubated at 37° C. for 24 h to obtain mature biofilms and washed with Hayflick to remove free cells. The preformed biofilms were treated (100 μL/well) with WT. WT-DispB, CV2 or CV2-DispB cell suspensions containing ˜1×109 CFU/mL or a similar volume of the culture supernatants of these strains filtered by 0.2 μm filters. As a control to normalize all the treatments, some wells were treated with Hayflick. Also, a staining control was carried out in wells in which no S. aureus biofilms were formed. After 15 min, 4 h, 8 h or 24 h of incubation at 37° C., wells were stained with crystal violet 0.1% (15 min, room temperature), washed and air dried. The crystal violet attached to the biofilm was solubilized with 100 μl/well of ethanol: acetone (80:20, vol/vol) and quantified by reading the absorbance at 595 nm (OD 595 nm) in a Multiscan microplate reader.

In Vitro P. aeruginosa Biofilm Degradation Assay

M. pneumoniae spp. was grown in a T25 flask for three days with 5 ml of Hayflick media without antibiotics, and then the conditioned supernatant was filtered with 0.33 μm sterile syringe filters. P. aeruginosa strains were grown overnight in Erlenmeyer flasks (20 μl stock in 20 ml TSB) at 37° C. on shaking 600 rpm, and then diluted to an OD600 of 0.15 in TSB. Diluted Pseudomonas culture (100 μl) was then added in triplicates to sterile 96-well polystyrene microtiter plates. Cells were incubated statically overnight at 25°° C. to allow for biofilm formation. Biofilms were washed with PBS the following day to remove non-adherent cells and TSB media. Treatment of 50-100 pl of M. pneumoniae-conditioned filtered medium were added to the wells (using at least triplicates), and plates were incubated at 37° C. for 4 h. After incubation, wells were washed with PBS, stained with 150 μl of 0.1% (w/v) crystal violet for 10 min and washed three times with PBS. The dye was solubilized by addition of 100 μl of 95% (v/v) ethanol and incubated for 10 min. Absorbance was measured at 595 nm using a TECAN plate reader.

### In Vitro Antimicrobial Activity Test

The antimicrobial activities of the supernatant of M. pneumoniae strains expressing pyocins were tested in a growth curve. M. pneumoniae was grown in a T25 flask to confluence (3-4 days at 37° C., 5% CO2) with 5 ml of Hayflick media without antibiotics, and then the supernatant media were filtered with 0.33-um sterile syringe filters. P. aeruginosa strains were grown overnight in Erlenmeyer flasks (20 μl stock in 20 ml TSB) at 37° C. with shaking, and then diluted to an OD600 of 0.1 in TSB. Diluted Pseudomonas culture (180 μl) was mixed with 20 μl of filtered M. pneumoniae supernatant into sterile 96-well polystyrene microtiter plates. All conditions were tested at least in triplicate. Plates were incubated in a TECAN reader at 37° C. with shaking, and absorbance was measured at 600 nm every 20 min.

### Western Blot Analysis

The M. pneumoniae strains were lysated in 4% SDS and quantified by BCA assay. 3 ug of each lysate were run on a 4-12% bis-tris protein gels and transferred to a nitrocellulose membrane with an iBlot western blot transfer (ThermoFisher). The membrane was blocked with 5% skim milk in 0.1% TBS-Tween and incubated overnight with the indicated antibodies. The antibodies used were produced by Proteogenix and used in 1:1000 dilution. Anti-rabbit HRP-conjugated IgG were used as secondary antibody. The chemiluminescent signal was acquired with an iBright Imaging System (ThermoFisher).

1. A genetically modified Mycoplasma bacterium comprising:
in its genome a deletion, substitution, and/or insertion of one or more nucleotides in the operons of the Ca2+ dependent cytotoxic nuclease gene (MPN133) or orthologues thereof and ADP-ribosyltransferase CARDS gene (MPN372) or orthologues thereof, that reduce the pathogenicity and/or immunogenicity of the genetically modified Mycoplasma bacterium as compared to a reference M129-B7 Mycoplasma pneumoniae bacterium,
the reduction in pathogenicity and/or immunogenicity being characterized by a reduction of toxicity by at least 30% upon introduction into a host organism when compared to the reference M129-B7 Mycoplasma pneumoniae bacterium,
wherein the genetically modified Mycoplasma bacterium further comprises in its genome an oligonucleotide arrangement, said oligonucleotide arrangement comprising:
i) a first nucleotide sequence encoding a first heterologous exopolysaccharide hydrolyzing enzyme under the control of a promoter or a functional variant of said the promoter or fragment thereof which is active in the genetically modified Mycoplasma bacterium, and wherein the exopolysaccharide hydrolyzing enzyme is Dispersin B, and
ii) at least one further nucleotide sequence encoding further heterologous exopolysaccharide hydrolyzing enzymes under the control of a promoter or a functional variant of the promoter or fragment thereof which is active in the genetically modified Mycoplasma bacterium, and
wherein the further exopolysaccharide hydrolyzing enzymes are selected from the group consisting of: Alginate lyase AI-II′, PelAh, PslGh, or any combination thereof.

2. The genetically modified Mycoplasma bacterium o of claim 1, wherein the genetically modified Mycoplasma bacterium further comprises one or more antimicrobial agents not encoded by or expressed in the reference Mycoplasma pneumoniae bacterium.

3. The genetically modified Mycoplasma bacterium according to claims 1, wherein the genetically modified bacterium is comprised in a pharmaceutical dosage unit along with one or more antimicrobial agents not encoded by or expressed in the reference Mycoplasma pneumoniae bacterium.

4. The genetically modified Mycoplasma bacterium of claim 2, wherein the antimicrobial agent is a bacteriocin, an antimicrobial peptide, an antibiotic, a defensin, or any combination thereof.

5. The genetically modified Mycoplasma bacterium of claim 4, wherein the bacteriocin is selected from the group consisting of: acidocin, actagardine, agrocin, alveicin, aureocin, aureocin A53, aureocin A70, bisin, camocin, camocyclin, caseicin, cerein, circularin A, colicin, curvaticin, divercin, duramycin, enterocin, enterolysin, epidermin, gallidermin, erwiniocin, gardimycin, gassericin A, glycinecin, halocin, haloduracin, klebicin, lactocin S, lactococcin, lacticin, leucoccin, lysostaphin, macedocin, mersacidin, mesentericin, microbisporicin, microcin, microcin S, mutacin, nisin, paenibacillin, planosporicin, pediocin, pentocin, plantaricin, pneumocyclicin, pyocin, reutericin 6, reutericyclin, reuterin, sakacin, salivaricin, sublancin, subtilin, sulfolobicin, tasmancin, thuricin 17, trifolitoxin, variacin, vibriocin, wamericin, wamerin, and any combination thereof.

6. The genetically modified Mycoplasma of claim 4, wherein the antibiotic is a bacterial membrane targeting antibiotic.

7. The genetically modified Mycoplasma bacterium of claim 1, wherein the Mycoplasma bacterium is Mycoplasma pneumoniae.

8. The genetically modified Mycoplasma bacterium of claim 1, wherein the first exopolysaccharide hydrolyzing enzyme is active against Pseudomonas aeruginosa and the at least one further exopolysaccharide hydrolyzing enzyme is active against Staphylococcus aureus.

9. (canceled)

10. A method of treating pneumonia, the method comprising: administering to the subject the genetically modified Mycoplasma bacterium of claim 1.

11. A method for increasing the in vivo potency of an attenuated bacterium as a therapeutic agent, the method comprising, administering the attenuated bacterium wherein the attenuated bacterium further comprises or expresses one or more antimicrobial agents.

12. The method according to claim 11, wherein the attenuated bacterium is a bacterium wherein one or more virulence factors are no longer functionally expressed.

13. The method according to claim 11, wherein the attenuated bacterium is a Mycoplasma pneumoniae bacterium.

14. The method according to claims 11, wherein the attenuated bacterium agent is active as a therapeutic agent against a bacterial infection.

15. The method according to claim 11, wherein the antimicrobial agent is lysostaphin.

16. The genetically modified Mycoplasma bacterium of claim 2, wherein the one or more antimicrobial agents are encoded by the genome of the genetically modified Mycoplasma bacterium.

17. The genetically modified Mycoplasma bacterium of claim 5, wherein the bacteriocin is lysostaphin.

18. The genetically modified Mycoplasma bacterium of claim 6, wherein the antibiotic is selected from the group consisting of: Piperacillin, Tazobactam, Ciprofloxacin, Levofloxacin, Meropenem, Imipenem, Cilastatin, Amikacin, Ceftazidime, Avibactam, Ceftolozane, Ceftriaxone, Vancomycin, Linezolid, and any combination thereof.

19. The method according to claim 10, wherein the pneumonia is ventilator-associated pneumonia.

20. The method according to claim 13, wherein the attenuated bacterium is a Mycoplasma pneumoniae bacterium and wherein the one or more virulence factors are Ca2+ dependent cytotoxic nuclease gene (MPN133) or orthologues thereof, and/or ADP-ribosyltransferase CARDS gene (MPN372) or orthologues thereof.

21. The method according to claim 14, wherein the bacterial infection characterized by biofilm formation due to the presence of Pseudomonas aeruginosa and/or Staphylococcus aureus.
